(19) United States
(12) Patent Application Publication
(10) Pub. No.: US 2002/0044948 A1
(43) Pub. Date: Apr. 18, 2002

(54) Methods and compositions for co-stimulation of immunological responses to peptide antigens

(76) Inventors: Samir Khleif, Silverspring, MD (US); Jay Berzofsky, Bethesda, MD (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/810,310

(22) Filed: Mar. 14, 2001

Related U.S. Application Data
(60) Provisional application No. 60/189,396, filed on Mar. 15, 2000.

(51) Int. Cl. A61K 39/00; A61K 39/38; A61K 39/02; C07K 1/00; C07K 14/00; C07K 17/00

(52) U.S. Cl. 424/234.1; 424/184.1; 530/350

(57) ABSTRACT

Method for eliciting an immune response in a vertebrate subject are provided involving administration of a peptide antigen to the subject in a coordinated vaccination procedure that also involves administration of a non-viral vector that encodes a T cell co-stimulatory molecule. The peptide antigen contains at least one T cell epitope and may include an epitope of a tumor antigen or an antigen of a viral or non-viral pathogen. Epitopes from tumor antigens may represent fragments or partial amino acid sequences of p53, ras, rb, mcc, apc, dcc; nfl; VHL; MEN1, MEN2, MLM, Her-2neu, CEA, PSA; Muc1, Gp100, tyrosinase, or MART1 proteins, and often span a mutation identified in the tumor antigen. Various viral antigens may be selected, for example antigens identified in a human immunodeficiency virus (HIV), hepatitis B virus (HBV), herpes simplex virus (HSV) or human papilloma virus (HPV), for production of peptide antigens corresponding to immunogenic epitopes of the viral antigen. The peptide antigen is administered simultaneously or sequentially with administration of the vector encoding the co-stimulatory molecules. Co-stimulatory molecules useful for coordinate administration with peptide antigens to elicit an enhanced T cell-mediated immune response may be selected from B7-1, B7-2, B7-3, ICAM1, ICAM2, LFA1 or LFA2. The peptide antigen and non-viral vector encoding the T cell co-stimulatory molecule are administered to proximal target sites selected from the same, or closely-adjacent, intradermal, subcutaneous, mucosal or intratumoral sites.

### CROSS-REFERENCES TO RELATED APPLICATIONS

This application claims the benefit of U.S. Provisional Patent Application No. 60/189,396, filed by Samir N. Khleif, et al., on Mar. 15, 2000.

### BACKGROUND OF THE INVENTION

For most viruses and non-viral pathogens, the greatest levels of host immunity are stimulated by natural infection. For viruses in particular, attempts to artificially elicit high-level host immunity frequently involve the use of live attenuated vaccines. However, such live attenuated viruses may be poorly suited for uninfected human recipients. Particularly in the case of retroviruses, such as HIV, inoculation of the attenuated vaccine organism may impose substantial risks, including possible integration of viral genome into the host cellular chromosomes and induction of immune disorders.

To reduce these risks, an alternative vaccination method is to use pure, well-characterized proteins or synthetic peptides as immunogens to elicit humoral and/or cellular immunity. It is well recognized that synthetic peptides incorporating immunogenic epitopes of a native pathogen can induce antibodies reactive with their cognate sequences in the native proteins. Likewise, several studies have reported the ability to immunize mammalian hosts with peptides to induce virus-specific or bacteria-specific cytotoxic T lymphocytes (CTLs) (Aichele et al., J. Exp. Med. 171:1815-1820, 1990; Schulz et al., Proc. Natl. Acad. Sci. USA 88:991-993, 1991; Kast et al., Proc. Natl. Acad. Sci. USA 88:2283-2287, 1991; Harty et al., J. Exp. Med. 175:1531-1538,1992; Hart et al., Proc Natl. Acad. Sci. USA 88:9448-9452, 1991; U.S. Pat. No. 4,599,230, and U.S. Pat. No. 4,599,231, each incorporated herein by reference).

CTLs play an essential role in fighting cells infected with viruses and other pathogens, as well as tumor cells. They do so by direct cytotoxicity and by providing specific and nonspecific help to other immunocytes such as macrophages, B cells, and other T cells. Infected cells or tumor cells process antigen through intracellular events involving proteases. The processed antigen is presented on the cellular surface in the form of peptides bound to HLA class I molecules to T cell receptors on CTLs. MHC class I molecules can also bind exogenous peptides and present them to CTLs without intracellular processing. Thus, numerous peptide-based vaccines aimed at eliciting CTL responses for prophylaxis or treatment of pathogenic infection and cancer have been proposed.

Many neoplasms express foreign antigens that can potentially serve as targets for destruction by the immune system (Hellstrom et al., in The Biologic Therapy of Cancer, pp. 35-52, Devita et al., eds., Philadelphia, J. B. Lippincott Co., 1991; and Boon, T., Adv. Cancer Res. 58:177-211, 1992, each incorporated herein by reference). Cellular immunity plays the key role in this rejection, with both T helper cells and cytotoxic T lymphocytes (CTLs) involved (Greenberg, P. D., Adv. Immunol. 49:281-355, 1991; and Melief, C. J. M., Adv. Cancer Res. 58:143-175, 1992, each incorporated herein by reference). Malignant transformation of a cell is commonly associated with phenotypic changes in the cell. Such changes can include loss or gain of expression of certain proteins or alterations in the level of expression of proteins. In conjunction with these changes, the immune system may recognize a tumor as foreign and mount an immune response against the tumor (Kripke, Adv. Cancer Res. 34:69-75, 1981; and Schreiber et al., Ann. Rev. Immunol. 6:465-483, 1988, each incorporated herein by reference). Consistent with this model, T cells have been identified which recognize and respond to tumor associated antigens presented by MHC molecules. Such tumor antigen-specific T lymphocytes have been demonstrated in the immune repertoire and are capable of recognizing and stimulating an immune response against tumor cells when properly stimulated in vitro. (Rosenberg et al., Science 233:1318-1321, 1986a; and Rosenberg et al. Ann. Rev. Immunol. 68:1-709, 1986b, each incorporated herein by reference).

However, in practice, tumors in vivo have generally not been found to be highly immunogenic and often appear capable of evading immune responses. This is attributed in part to an inability of tumor cells to induce T cell-mediated immune responses. (Ostrand-Rosenberg, et al., J. Immunol. 144:4068-4071, 1990; and Fearon, E. R. et al., Cell 60:397-403, 1990, each incorporated herein by reference). Consistent with this model, many tumors fail to adequately process and present antigens to T cells. This is thought to be due, at least in part, to reduced expression of MHC class I molecules in tumor cells (Elliot at al., Adv. Cancer Res. 53:181-244, 1989, incorporated herein by reference). Lack of an effective antitumor immune response may also result from a deficiency of T helper functions in tumor-bearing animals. In this context, transfection of tumor cells with interleukin 2 (IL-2) or interleukin 4 (IL-4) cDNAs reportedly facilitate paracrine secretion of lymphokines that can substitute for T cell help, induce tumor-specific CTLs, and cause tumor rejection (Fearon et al., supra; Gansbacher et al., J. Exp. Med. 172:1217-1224, 1990b; Ley et al., Eur. J. Immunol. 21:851-854, 1991; and Golumbek et al., Science 254:713-716, 1991, each incorporated herein by reference).

Another mechanism that may contribute to the inefficient induction of tumor-reactive T cells is suggested by the “two-signal” model for lymphocyte activation. This model was originally proposed for B lymphocytes (Bretscher and Cohn, Science 169:1042-1049, 1970, incorporated herein by reference) and later as an explanation for why antigens expressed on cells of nonhematopoetic origin are ineffective at inducing transplant rejection (Lafferty et al., Ann. Rev. Immunol. 1:143-173, 1983, incorporated herein by reference). A two-signal model has now been proposed for all lymphocytes (Janeway, C. A., Jr., Cold Spring Harbor Ouant. Biol. 54:1-13, 1989; Nossal, G. J. V., Science 245:147-153, 1989; and Schwartz, Cell 57:1073-1081, 1989, each incorporated herein by reference). According to this model, optimal stimulation and effective antigen-specific clonal expansion of lymphocytes require both a primary, antigen-specific signal, and a secondary, “co-stimulation” signal.

The primary activation signal typically involves an antigenic peptide bound either to a major histocompatibility complex (MHC) class I protein or a MHC class II protein present on the surface of an antigen presenting cell (APC). CD4+ T cells recognize peptides associated with class II molecules. Class II molecules are found on a limited number of cell types, primarily B cells, monocytes/macrophages and dendritic cells, and, in most cases, present peptides derived from proteins taken up from the extracellular environment. In contrast, CD8+ T cells recognize peptides associated with class I molecules. Class I molecules are found on almost all cell types and, in most cases, present peptides derived from endogenously synthesized proteins (see, e.g., Germain, R., Nature 322:687-691, 1986, incorporated herein by reference).

T cell co-stimulation is neither antigen-specific, nor MHC restricted, and is thought to be provided by one or more distinct cell-surface molecules expressed by antigen presenting cells (APCs) (Jenkins et al., J. Immunol. 140:3324-3330, 1988; Linsley et al., J. Exp. Med. 173:721-730, 1991; Gimmi et al., Proc. Natl. Acad. Sci. USA 88:6575-6579, 1991; Young et al., J. Clin. Invest. 90:229-237, 1991; Koulova et al., J. Exp. Med. 173:759-762, 1991; Reiser et al., Proc. Natl. Acad. Sci. USA 89:271-275, 1992; van-Seventer et al., J. Immunol. 144:4579-4586, 1990; LaSalle et al., J. Immunol. 147:774-780, 1991; Dustin et al., J. Exp. Med. 169:503, 1989; Armitage et al., Nature 357:80-82, 1992; and Liu et al., J. Exp. Med. 175:437-445, 1992, each incorporated herein by reference). More specifically, co-stimulation is believed to involve binding of co-stimulatory molecules on the surface of the antigen presenting cells to a corresponding T cell-ligand (see, e.g., Mueller et al., Annu. Rev. Immunol. 7:445-480, 1989, incorporated herein by reference).

Considerable evidence suggests that the B7 protein, expressed on APCs, is one such co-stimulatory molecule (Linsley et al., 1991, supra; Gimmi et al., 1991, supra; Koulova et al., supra; Reiser et al., supra; Linsley et al., Proc. Natl. Acad. Sci. USA 87:5031-5035, 1990; and Freeman et al., J. Exp. Med. 174:625-631, 1991, each incorporated herein by reference). B7 is a counter-receptor for two ligands expressed on T lymphocytes. The first ligand, termed CD28, is constitutively expressed on resting T cells and increases after activation. After signaling through the T cell receptor, ligation of CD28 induces T cells to proliferate and secrete IL-2 (Linsley, 1991, supra; Gimmi, 1991, supra; Thompson et al., Proc. Natl. Acad. Sci. USA 86:1333-1337, 1989; June et al., Immunol. Today 11:211-216, 1990; and Harding et al., Nature 356:607-609, 1992, each incorporated herein by reference). The second ligand, termed CTLA4 is highly homologous to CD28 but is not expressed on resting T cells and appears following T cell activation (Brunet et al., Nature 328:267-270, 1987, incorporated herein by reference).

The importance of the B7:CD28/CTLA4 co-stimulatory pathway has been further evaluated using in vitro and in in vivo model systems. Studies using CTLA-4-deficient mice and CTLA-4-blocking monoclonal antibodies suggest that CTLA-4 signaling negatively affects T cell responses. As evidence of this function, CTLA-4 knockout mice exhibit spontaneous autoimmune disease. In contrast, blockade of CD28-B7 co-stimulation reportedly results in development of antigen specific tolerance in murine and human systems (Harding, et al., 1992, supra; Lenschow et al., Science 257:789-792, 1992; Turka et al., Proc. Natl. Acad. Sci. USA 89:11102-11105, 1992; and Gimmi et al., Proc. Natl. Acad. Sci USA 90:6586-6590, 1993, each incorporated herein by reference). In related studies, transfection of B7-negative murine tumor cells, using a viral vector to express B7, reportedly induces T-cell mediated immunity, accompanied by tumor rejection and protection against subsequent tumor challenge (Chen et al., Cell 71:1093-1102, 1992; Townsend et al., Science 259:368-370, 1993; and Baskar et al., Proc. Natl. Acad. Sci. 90:5687-5690, 1993, each incorporated herein by reference). In related studies, Freeman et al., U.S. Pat. Nos. 5,942,607 and 5,861,310 (each incorporated herein by reference), describe two additional counter-receptors related to B7 (hereafter “B7-1”), designated B7-2 and B7-3. Transfection of murine sarcoma cells with all three B7 clones using a viral vector reportedly was effective to confer immunogenicity on the modified cells when used as an immunogen in A/J mice, whereby the B7-transfected tumor cells reportedly elicited protection against subsequent challenge with malignant tumor cells.

Despite the foregoing advances in the art of immunotherapy to treat pathogenic infection and cancer, there remains a clear need for additional tools to supplement and enhance existing vaccines and treatment methods. The use of peptide immunogens to treat or prevent viral infections and cancer holds considerable promise. However, additional developments are needed to enhance the efficacy of peptide vaccines and extend their application to a broader range of clinical settings. Toward this end, the use of viral vectors encoding co-stimulatory factors such as B7 has been proposed as a separate intervention for immnunotherapeutic treatment. However, questions remain regarding the precise roles of B7 and other co-stimulatory agents in mediating or enhancing T-cell activation. At the same time, introduction of co-stimulatory factors using viral vectors is attended by inherent biological risks, as well as deficiencies such as antigenicity of the viral vector in the immunized host. Thus, the use of viral vectors encoding B7 as an irmmunotherapeutic remains of uncertain clinical value for treating viral infection and cancer. Alternative methods that might rely on delivery of full-length co-stimulatory proteins have not been successfully implemented. In any event, such methods would need to overcome inherent problems that attend full-length protein delivery, including a requirement for in vivo stability and conformational integrity of the protein, as well as proper presentation of the co-stimulatory factor to achieve T-cell activation after exogenous delivery.

Surprisingly, the methods and compositions of the instant invention fulfill the foregoing needs and advantages by supplementing and enhancing peptide-based vaccines and treatment methods. At the same time, the invention obviates specific problems that attend previously reported methods for eliciting T-cell co-stimulation. The methods and compositions of the invention satisfy these objects and fulfill additional objects and advantages that will become apparent from the description which follows.

### SUMMARY OF THE INVENTION

This present invention provides methods and compositions for supplementing and enhancing peptide- and protein-based vaccines and treatment methods. The methods of the invention involve coordinate administration of an immunogenically effective amount of a peptide or protein antigen and a non-viral vector encoding one or more T cell co-stimulatory molecules. The peptide or protein antigen and vector encoding the co-stimulatory molecule may be delivered together as a mixture, or separately in a sequential vaccination protocol, and are typically combined with a pharmaceutically acceptable diluent or carrier. The peptide or protein antigen may be administered to the subject by incorporating the antigen in a vaccine formulation as a purified protein or a tumor lysate component of the vaccine formulation.

In specific aspects of the invention the peptide or protein antigen includes at least one T cell epitope, for example a T helper cell epitope and/or a cytotoxic T cell (CTL) epitope, capable of eliciting a specific T cell response in the subject. The antigen is coordinately administrated with a non-viral vector that incorporates a polynucleotide encoding one or more co-stimulatory molecules, wherein the vector is effective to direct expression of the co-stimulatory molecule(s) in target cells of the subject. In certain embodiments, multiple co-stimulatory molecules are encoded and expressed by the vector, or multiple vectors encoding multiple co-stimulatory molecules are administered coordinately with the peptide antigen. The antigen and vector are preferably delivered to proximal target sites, for example to the same or closely-adjacent intradermal, subcutaneous, mucosal site, or intratumoral site.

The non-viral vector may comprise a RNA or DNA vector. In exemplary embodiments, the vector comprises a naked DNA vector that incorporates the polynucleotide encoding the co-stimulatory molecule operably linked to regulatory elements necessary for expression of the co-stimulatory molecule in eukaryotic cells.

In alternative aspects of the invention, the peptide or protein antigen may incorporate a T cell epitope of a tumor antigen or antigen of a viral or non-viral pathogen. In more detailed aspects, the peptide or protein antigen incorporates an epitope from a tumor antigen, for example the proteins (or products encoded by) p53, ras, rb, mcc, apc, dcc, nfl, VHL, MEN1, MEN2, MLM, Her-2neu, CEA, PSA, Muc1, Gp100, tyrosinase, and MART1. Alternatively, the peptide or protein antigen may comprise an epitope of a viral antigen, for example an antigen of human immunodeficiency virus (HIV), hepatitis B virus (HBV), hepatitis C virus (HBC), herpes simplex virus (HSV) or human papilloma virus (HPV).

Within the methods and compositions of the invention, co-stimulatory molecules are selected for their ability to stimulate T cell activation (e.g., as marked by T cell proliferation and/or increased cytokine production and/or the ability to lyse target cells). In addition, co-stimulatory molecules are selected that enhance antigen specific immune responses in the subject when coordinately administered with a peptide or protein antigen. Exemplary co-stimulatory molecules for use within the invention include B7-1, B7-2, B7-3, B7H, ICAM1, ICAM2, ICAM3; LFA1, LFA2, and LFA3.

Within additional aspects of the invention, immunogenic compositions are provided that include an immunogenically effective amount of a peptide or protein antigen possessing at least one T cell epitope, and a non-viral vector incorporating a polynucleotide that encodes a T cell co-stimulatory molecule operably linked to regulatory elements necessary for expression of the co-stimulatory molecule in eukaryotic cells. The peptide or protein antigen and vector are admixed in a pharmaceutically acceptable carrier or diluent for delivery to the subject.

### BRIEF DESCRIPTION OF THE DRAWINGS

FIG. 1 illustrates the co-stimulatory effects of CTL activity elicited by coordinate administration of a B7-1-encoding vector with an HPV E7 peptide antigen, as compared with the immunogenic response elicited by the E7 peptide antigen alone. C57B1/6 mice were vaccinated with emulsion containing E7 peptide with or without co-administration of a B7.1-encoding DNA vector. B7.1 DNA was administered, either in the same site as the E7 peptide or on the other side of the base of the tail, as indicated. The FIGURE shows that coordinate administration of the B7.1-encoding DNA vector significantly enhances the E7-specific CT1 response when the vector and peptide are delivered to a common target site.

### DESCRIPTION OF THE SPECIFIC EMBODIMENTS

The novel methods and compositions of the invention combine effective aspects of peptide vaccination with co-stimulation of T cell immunity. Co-stimulation of T-cells is achieved by coordinate immunization with a peptide or protein antigen and a non-viral vector encoding a co-stimulatory molecule. In conjunction with peptide immunization, administration of the non-viral vector encoding the co-stimulatory molecule elicits an enhanced, antigen-specific immune response, i.e., as compared to the immune response elicited by peptide or protein antigen alone. Administration of co-stimulatory molecules via a non-viral vector provides for improved delivery over direct protein delivery or delivery using viral vectors.

In particular, delivery of co-stimulatory molecules by the methods of the invention overcomes inherent problems of many viral vectors, including biologic risks, requirements for proper viral particle assembly, and potential antigenicity of the vector. Commonly used retroviral vectors have restrictions on the size and structure of polypeptides that can be expressed while maintaining the ability of the recombinant virus to replicate, and the effectiveness of vectors such as vaccinia for subsequent immunizations may be compromised by immune responses against the vectors themselves. Further, in contrast to other types of full-length protein delivery systems, the methods and compositions of the invention are useful in combination with other types of vaccines, including tumor lysates, whole tumor cell preparations, recombinant proteins and DNA vectors. In addition, the methods and compositions of the invention are inexpensive and easy to produce in comparison to other vaccine methods and preparations.

As noted above, the invention provides novel methods and compositions to supplement and enhance peptide-based vaccines to prevent and/or treat pathogenic infections and cancer. The methods of the invention involve coordinate administration of an immunogenically effective amount of a peptide or protein antigen and a non-viral vector encoding a T cell co-stimulatory molecule. The peptide or protein antigen and vector encoding the co-stimulatory molecule are preferably delivered to proximal target sites, for example to the same or closely-adjacent intradernal, subcutaneous, mucosal site, or intratumoral site.

In specific aspects of the invention the peptide or protein antigen includes at least one T cell epitope, for example a T helper cell epitope and/or a cytotoxic T cell (CTL) epitope, capable of eliciting a specific T cell response in the subject. T lymphocytes can recognize antigens presented by major histocompatibility complex (MHC) class I or class II molecules (Brodsky et al., Tissue Antigens 47:464-471, 1996, incorporated herein by reference). These surface antigens are composed of short peptides, ordinarily derived from degraded intracellular proteins (Townsend et al., Ann. Rev. Immunol. 7:601-624, 1989; and Rotzschke et al., Nature 348:252-254, 1990, each incorporated herein by reference). CD8+ T cells recognize peptides (8-10 residues) bound to MHC class I molecules and are associated with cytotoxic activity (Eisenlohr et al., J. Immunol. Meth. 154:131, 1992; and Rock et al., Prod. Natl. Acad. Sci. USA 89:8918-8922,1992, each incorporated herein by reference). The CD4+ lymphocytes recognize peptides (13-18 residues) presented by MHC class II molecules (Hunt et al., Science 256:1817-1820, 1992; and Rudensky et al., Nature 353:622-627, 1991, each incorporated herein by reference). They are involved in immunoregulation through cytokine secretion and, in some subtypes, can also exhibit cytotoxic activity (McKisic et al., J. Immunol. 150:3793, 1991; Ozdemirli et al., J. Immunol. 149:1889-1895, 1992; and Chang et al., J. Immunol. 145:409, 1990, each incorporated herein by reference). Within the methods of the invention, peptide or protein antigens and vectors for expressing co-stimulatory molecules provide primary and co-stimulatory signals to stimulate effective immune responses from either CD4+ or CD8+ T cells, or both cell types.

T lymphocytes play an essential role in fighting cells infected with viruses and other pathogens, as well as tumor cells. They do so by direct cytotoxicity and by providing specific and nonspecific help to other immunocytes such as macrophages, B cells, and other T cells. Infected cells or tumor cells process antigen through intracellular events involving proteases. The processed antigen is presented on the cellular surface in the form of peptides bound to HLA class I molecules to T cell receptors on CTLs. MHC class I molecules can also bind exogenous peptides and present them to CTLs without intracellular processing. Thus, numerous peptide vaccines aimed at eliciting CTL responses for prophylaxis or treatment of pathogenic infection and cancer have been proposed, and many such vaccines are useful within the peptide immunization/co-stimulation methods of the invention.

In addition to treating and/or prevention infections and cancer, the methods of the invention are useful for treating certain autoimmune disorders. In this regard, Sun and coworkers (Nature 332:843, 1988; and Eur. J. Immunol. 18:1993, 1988, each incorporated herein by reference) have reported the isolation of cytotoxic T cell clones specific for idiotypic determinants on encephalitogenic T cells, which adoptively transfer resistance to experimental autoimmune encephalomyelitis. The concept of immunization of subjects with autoimmune disease with immunogens that would induce an immune response against the autoimmune clone of T cells has recently been recognized as an accepted vaccination approach to treat or prevent autoimmune responses (reviewed by Cohen et al., Immunol. Today 332, 1988; Howell et al., Science 246:668, 1989; and Wralth et al., Cell 57:709, 1989, each incorporated herein by reference).

Thus, the present invention provides a simple and effective method for inducing and co-stimulating MHC-restricted Class I or Class II cytotoxic T cells using peptides corresponding to host antigens, and thus represents a significant advance in the development of vaccines for autoimmune disease. Using standard recombinant DNA techniques and existing probes and sequences for T cell receptor (TCR) molecule antigen binding regions, sequences can be obtained from unique regions of the TCR molecules (Barns et al., J. Exp. Med. 169:27, 1989, incorporated herein by reference). These peptides, administered coordinately with a vector for expressing a co-stimulatory molecule, can be used to induce an enhanced cytotoxic T cell immune response targeted against specific clones of T cells bearing TCRs responsible for antigen-specific T cell-mediated host tissue damage. Thus induced, the anti-TCR-targeted cytotoxic T cell response can eliminate autoreactive cells, thereby providing a novel, highly specific strategy for the control of T cell-mediated tissue destruction.

Another example of the use of peptide vaccine/co-stimulation methods of the invention is to control antibody-mediated tissue damage that occurs in the context of autoimmune diseases, infectious diseases, and in the setting of organ transplantation. B cell surface receptors for antigen (surface immunoglobulin) also contain regions that are specific for clones of B cells making antibodies. By identifying clones of B cells producing antibodies responsible for tissue-specific damage in the setting of the above disease categories, the sequence of peptides from the region of the B cell immunoglobulin molecule that binds antigen can be identified using, for example, recombinant DNA techniques. Further evaluation of these sequences will identify peptide or protein antigen sequences capable for use within the compositions and methods of the invention for inducing MHC Class I or Class II cytotoxic T cell responses. These peptides, administered coordinately with a vector for expressing a co-stimulatory molecule, can thus be used to induce an enhanced cytotoxic T cell immune response targeted against autoantibody-producing B cells, thereby preventing or inhibiting tissue-damaging autoantibody responses that occur in the context of the above disease categories.

Yet another example for using peptide vaccine/co-stimulation methods of the invention is directed toward specific elimination of autoreactive T and B cell types for the treatment of clonal B and T cell malignancies that express on their surface clonal immunoglobulin or TCR molecules. Anti-tumor therapeutic strategies have been described that employ antibodies against variable regions of either B cell surface immunoglobulin molecules (Hamblin et al., Brit. J. Cancer 42:495, 1980; and Miller et al., N. Ens. J. Med. 306:517, 1982, each incorporated herein by reference) or antibodies against variable TCR regions in the case of treatment of T cell tumors (Kanagawa, J. Exp. Med. 170:1513-1519, 1989, incorporated herein by reference). Thus, peptide or protein antigens containing the sequences of variable regions of the TCR or immunoglobulin molecules expressed on the surface of T or B cell malignant cells, respectively, can be administered to the subject coordinately with a vector encoding a co-stimulatory molecule to induce anti-TCR or anti-immunoglobulin-specific cytotoxic T cell responses that kill the malignant T or B cells.

In more detailed embodiments of the invention, the peptide or protein antigen for coordinate administration with the vector encoding a co-stimulatory molecule may incorporate one or more T cell epitopes of a tumor antigen. Present theories of tumor initiation and progression hold that tumor cells typically arise from mutational events, either inherited or somatic, that occur in a normal cell. These events lead to escape from normal control of proliferation in the cell population which contains the tumorigenic mutation(s). Almost all tumors express genes whose products are required for malignant transformation or the maintenance of the malignant phenotype. In many instances these genes are altered forms of normal cellular genes that are important for the control of cell division and differentiation. Exemplary tumor antigens (or the genes encoding the antigens) which have been identified as exhibiting mutations associated with human cancers include p53 (lung, colon and breast cancer, Li-Fraumeni syndrome), rb (retinoblastoma, small cell lung cancer), mcc, apc (colon cancer, familial polyposis, Gardner's syndrome), dcc (colon cancer); nfl (neurofibromatosis); VHL (renal cell carcinoma); MEN2 (multiple endocrine neoplasia, type 1); MEN1 and MEN2 (multiple endocrine neoplasia); MLM (familial melanoma, lung cancer); ras (solid tumors); Her-2neu (breast and ovarian cancer), carcinoembryonic antigen (CEA); Prostate specific antigen (PSA); Mucd (adenocarcinomas), as well as Gp100, tyrosinase, and MART1 (melanoma). Additional peptide antigens for use within the invention have an amino acid sequence that encompass a fusion joint of a tumor-specific fusion protein encoded by a human chromosomal translocation. Two preferred fusion protein targets for the immunization are created by the translocations t(11;22) (q24;q12) and t(2;3) (q35;q14) (U.S. patent application 08/472,298, filed Jun. 7, 1995 by Berzofsky et al., and corresponding International Publication No. WO 94/21287, incorporated herein by reference). Yet additional peptide antigens incorporate mutations that accumulate during initiation and progression of colon cancer, or are found in key growth control genes (Molecular Foundations of Oncology, S. Broder, ed. c. 1991 by Wilkins and Wilkins, Baltimore, Md). Peptides spanning both normal and mutant portions of these and other tumor antigens (see, e.g., International Publication No. WO 92/14756, incorporated herein by reference) are readily produced (e.g., by automated peptide synthesis) and evaluated (e.g., by CTL cytolysis assay) for operability within the methods and compositions of the invention.

In specific embodiments of the invention, peptide or protein antigens are derived from mutant or normal tumor antigens selected from the oncogene ras and the tumor suppressor gene p53, both of which are commonly mutated in human cancers (20% and 50% respectively for all solid tumors). Processed peptide fragments of these altered proteins are presented in the context of major histocompatibility (MHC) molecules on the cell surface can be recognized by the immune system as foreign, thereby acting as tumor antigens. T-cell responses specific for mutated ras and p53 proteins have been demonstrated in mice and humans (Peace et al., J. Immunol. 146:2059, 1991; and Yanuck et al., Cancer Res. 53:3257, 1993, each incorporated herein by reference).

Ras is among the best characterized oncogenes in human cancers is ras (Boa et al., Nature 327:293-297, 1987; Satoh et al., Semin. Cancer Biol. 3:169-177, 1992; and Grand et al., Biochemistry 279:609-631, 1991, each incorporated herein by reference). A single point mutation in codon 12 of the ras gene accounts for more than 90% of all ras mutations (Hruban et al., Am. J. Pathol. 143:545-554, 1993; Li et al., Am. J. Pathol. 144:303-309, 1994; and Breivik et al., Br. J. Cancer 69:367-371, 1994, each incorporated herein by reference), and is present in more than 20% of all solid tumors (i.e., more than 800,000 cancer patients in the United States). In addition, the Ala⁵⁹, Gly⁶⁰ and Gln⁶¹ residues of the ras proto-oncogene are frequently mutated in human tumors (Chung et al., Science 259:806-809, 1992, incorporated herein by reference).

Therefore, mutant ras peptides serve as particularly useful vaccine agents to elicit anti-cancer immune responses according to the methods of the invention. In this context, Ras p21 is an intracellular protein subject to antigenprocessing and presentation by MHC molecules. Specific CD4+ and CD8+ T lymphocytes that can recognize a single ras mutation have been described. Murine experiments have shown that T lymphocytes specifically immunoreactive against mutated ras peptides have the ability to lyse target cells that endogenously express the same point mutated ras gene. These lytic T cells display cytotoxic activity of both CD4+ (Th1 subtype) and CD8+ subsets (Abrams et al., Eur. J. Immunol. 25:2588-2597, 1995; Peace et al., J. Immunother. 14:110-114, 1993; Peace et al., J. Exp. Med. 179:473-479, 1994; and Skipper et al., J. Exp. Med. 177:1493-1498, 1993, each incorporated herein by reference). Furthermore, induction of anti-Ras CTLs by vaccinating mice with recombinant mutant ras proteins has led to the rejection of syngeneic tumor cells bearing the corresponding mutation (Fenton et al., J. Natl. Cancer Inst. 85:1294-1302, 1993, incorporated herein by reference).

Human studies involving mutant ras peptides have shown that in vitro priming of peripheral blood mononuclear cells (PBMC) with the peptides generates specific CD4+ or CD8+ T-cell responses (Abrams et al., Eur. J. Immunol. 26:435-443, 1996; Fossum et al., Cancer Immunol. Immunother. 40:165-172, 1995; Fossum et al., Int. J. Cancer 56:40-45, 1994; Tsang et al., Vaccine Res. 3:183-193, 1994; Gedde-Dahl et al., Eur J. Immunol. 23:754-760, 1993; Van Elsas et al., Int. J. Cancer 61:389-396, 1995; and Jung et al., J. Exp. Med. 73:273-276, 1991, each incorporated herein by reference). It has also been shown that specific immune responses can be elicited in cancer patients against mutant ras by peptide vaccination (Gjertsen et al., Lancet 346:1399-1400, 1995; Gjertsen et al., Int. J. Cancer 72:784-790, 1997, each incorporated herein by reference). These studies also demonstrate that mutant ras protein sequences are capable of being presented on human tumor cells and recognized by specific human T cells. More recently a phase I pilot trial has been completed in which 13-mer mutant ras peptides were administered subcutaneously with the immune adjuvant (Detox) to cancer patients bearing the corresponding ras mutation in their tumors. In this study, vaccinated cancer patients tolerated different dose levels of up to 5,000 μg of the mutated ras peptides formulated with Detox (RiBi Immunochem Research, Hamilton, MT) as an adjuvant (Khleif et al., J. Immunother. 22:155-165, 1999, incorporated herein by reference). No serious acute or long-term side effects were observed. Additional in vivo studies are presently underway to generate or boost immune responses using other specific mutant ras peptides, as well as mutant p53 peptides, modeled after mutant proteins identified in patient tumors (see, e.g., Khleif et al., J. Immunother. 22:155-165, 1999; U.S. patent application 08/472,298, filed Jun. 7, 1995 by Berzofsky et al., and corresponding International Publication No. WO 94/21287, each incorporated herein by reference).

Various mechanisms in addition to spontaneous oncogene formation may generate tumor-associated antigens that can be perceived and targeted by the immune system, which antigens are therefore useful within the methods of the invention (Tsomides et al., Proc. Natl. Acad. Sci. USA 91:3487, 1994; and Pardoll, Nature 369:357, 1994, each incorporated herein by reference). Mechanisms that may provide for immune recognition of tumors include activation of a silent gene, overexpression or alteration of a normal gene, expression of oncofetal antigens, and the acquisition of viral oncogenes.

Activation of a silent gene can produce a new protein molecule in tumor cells that may not be previously recognized as self and, thus, may be antigenic for the naive immune system. An example of such a gene is MAGE1 in melanoma (van der Bruggen, P., Science 254:1643, 1991, incorporated herein by reference).

Overexpression of a normal gene can produce higher expression of a certain protein in tumor cells. This higher expression can overcome a low level of protein expression in normal tissue, which may otherwise escape the induction of immune tolerance. Molecules that are recognized through this mechanism may be oncogenic, for example, HER2/neu, or nononcogenic, for example, the melanoma antigens MARTI, gp100, and tyrosinase (Kawakami et al., J. Immunother. 14:88, 1993, incorporated herein by reference).

Expression of oncofetal antigens is another mechanism by which unique tumor-associated antigens can develop. Oncofetal antigens are proteins that are normally expressed on early fetal but not adult tissues. Thus, these proteins may have escaped the process by which lymphocytes recognize self. The proteins become expressed as a result of specific gene re-activation in tumor cells by an unknown mechanism. Carcinoembryonic antigen (CEA) is among the best characterized of this type of protein. CEA is expressed at very low levels in normal colonic epithelium, but is greatly overexpressed in colonic carcinoma cells. Recently, a CEA vaccine clinical trial has demonstrated generation of specific immunological response against the CEA protein in patients with colon cancer.

Viral oncogenesis is a well studied process that provides for anti-tumor immune recognition. Both RNA and DNA viruses are implicated in the development of some human cancers. Tumors that are induced by viruses usually contain integrated viral genome and often express viral proteins encoded by the viral genome. These endogenously synthesized proteins can be processed in a fashion analogous to other self proteins and presented as antigens on the surface of tumor cells. Human papilloma virus (HPV) is one such oncogenic virus that is associated with development of human cervical cancer. HPV-E6 and HPV-E7 proteins are two HPV proteins that have been shown to function as tumor antigens. These proteins therefore provide useful subjects for generation of peptide or protein antigens for use within the invention to generate T lymphocyte-mediated immune responses. Exemplifying these aspects of the invention, co-stimulation involving coordinate administration of an antigenic HPV16 E7 peptide (49-57) and a vector adapted to direct expression of co-stimulatory molecule B7 elicited a 3-4-fold greater CTL response than the E7 peptide alone.

In addition to cancer treatment methods, the invention also provides methods and compositions that elicit primary induction and co-stimulation of immune responses directed against viral and non-viral pathogens. Within this aspect of the invention, it is well recognized that synthetic peptides incorporating immunogenic epitopes of native pathogens can be used to immunize vertebrate subjects to induce virus-specific or bacteria-specific T cell responses, including cytotoxic T lymphocyte (CTL) responses (Aichele et al., supra; Schulz et al., supra; Kast et al., supra; Harty et al., supra; Hart et al., supra; U.S. Pat. No. 4,599,230, and U.S. Pat. No. 4,599,231, each incorporated herein by reference). Thus, peptide or protein antigens capable of eliciting specific T-cell responses against selected viral and non-viral pathogens are readily employed within the peptide vaccination/co-stimulation methods of the invention.

One pathogen of particular interest for prophylactic and therapeutic treatment according to the methods of the invention is the human immunodeficiency virus (HIV). Persons skilled in the art will readily appreciate from a reading of the present disclosure that MHC Class I-restricted cytotoxic T cells specifically targeted against HIV can be induced by administering in vivo a peptide or protein antigen comprising one or more CTL epitopes derived from or patterned after a sequence present in an HIV antigenic protein. Thus, within the methods and compositions of the invention, representative peptide or protein antigens comprising CTL epitopes directed against HIV are coordinately administered or combined in formulations with a vector for expressing a T-cell co-stimulatory molecule.

Exemplary peptides for eliciting specific immune responses against HIV to provide a primary signal within the co-stimulatory methods of the invention are disclosed by Berzofsky and colleagues (U.S. Pat. No. 5,939,074; U.S. Pat. No. 5,976,541; U.S. Pat. No. 5,030,449; U.S. Pat. No. 5,081,226; U.S. Pat. No. 5,932,218; U.S. Pat. application Ser. No. 08/060988, filed 14 May, 1993, corresponding to EP 0 710 572 BI; WO 94/26785; and U.S. Pat. application Ser. No. 09/455,076, each incorporated herein by reference). Within specific embodiments of the invention, the peptide antigen comprises a multideterminant cluster peptide of a HIV envelope protein, for example PCLUS 1, PCLUS 2, PCLUS 3, PCLUS 4, PCLUS 5, PCLUS 6, and PCLUS 6.1, as disclosed in the above-incorporated references. Also available for use within the invention are peptide or protein antigens that comprise a multideterminant cluster peptide of a HIV envelope protein and a second peptide, for example a P18 HIV peptide, that contains a T- or B-cell epitope. Thus, for example, a peptide antigen for use within the invention, for use against HIV or any other selected pathogen, may comprise a first peptide sequence having an epitope derived from a target antigen that is capable of eliciting a-helper T cell response, a second peptide sequence having an epitope capable of eliciting a cytotoxic T cell response, and a third peptide sequence having an epitope capable of eliciting a high titer neutralizing antibody response to the same viral antigen. Among these peptide or protein antigens, HIV-specific peptides may include second and third peptide sequences derived from the V3 loop of gp160 of HIV-1. Each of the three peptide sequences may be attached to both of the other peptide sequences in a contiguous manner. Exemplary peptide antigens within this aspect of the invention may include the following amino acid sequences:

| | |
|---|---|
| EQMHEDIISLWDQSLKPCVKRIQRGPGRAFVTIGK | (SEQ ID NO.1), |
| KQIINMWQEVGKAMYAPPISGQIRRIQRGPGRAFVTIGK | (SEQ ID NO.2), |
| RDNWRSELYKYKVVKIEPLGVAPTRJQRGPGRAFVTIGK | (SEQ ID NO.3), |
| AVAEGTDRVIEVVQGAYRAIRHIPRRIRQGLERRIQRGPGPA | (SEQ ID NO.4), |
| FVTIGK | |
| DRVIEVVQGAYRAIRHIPRIRQGLERRIQRGPGRAFVTIGK | (SEQ ID NO.5), |
| DRVIEVVQGAYRAIRRIQRGPGRAFVTIGK | (SEQ ID NO.6), |
| AQGAYRAIRHIPRIRRIQRGPGRAFVTIGK | (SEQ ID NO.7), |
| EQMHEDHSLWDQSLKPCVKAUHIGPGRAFYTTKN | (SEQ ID NO.8), |
| KQIINMWQEVGKAMYAPPISGQIRRHIGPGRAFYTTKN | (SEQ ID NO.9), |
| RDNWRSELYKYKVVKIEPLGVAPTRIHIGPGRAFYTTKN | (SEQ ID NO.10), |
| AVAEGTDRVIEVVQGAYRAIRHIPRHRQGLERRIHIGPG | (SEQ ID NO.11), |
| RAFYTTKN | |
| DRVIEVVQGAYRRIIIPRRIRQGLERPIHIGPGRAFYTTKIN | (SEQ ID NO.12), |
| DRVIEVVQGAYRAIRRIHIGPGRAIYTTKN | (SEQ ID NO.13), |
| or | |
| AQGAYRAIRHIPRRIRRIHIGPGRAFYTTKN | (SEQ ID NO.14). |

In more detailed embodiments of the invention, peptide or protein antigens for use within co-stimulatory compositions and methods are based on the HIV -1 (IIIB) gp160 envelope glycoprotein-derived peptide, p18. The prototype p18 peptide is 15 amino acids in length (residues 315-329 numbered according to Ratner et al. (Nature 313:277, 1985, incorporated herein by reference). It is the immunodominant CTL determinant of gp 160 in H-2D^{d} mice (Takahashi et al., Proc. Natl. Acad. Sci. USA 85:3105, 1988; and Takahashi et al., J. Exp. Med. 170:2023, 1989, each incorporated herein by reference) and can sensitize syngeneic cells for lysis by CTL from HIV-1-infected humans (Clerici et al., J. Immunol. 146:2214, 1991, incorporated herein by reference). Although it has generally been assumed that the same antigenic peptides are not presented by both class I and class II MHC molecules, there are a few cases reported in which peptides presented by class I molecules were found to be presented by or to bind to class II molecules also (Perkins et al., J. Exn. Med. 170:279, 1989; and Hickling et al., Internat. Irninunol. 2:435, 1990, each incorporated herein by reference). Among these cases, the P18IIIB peptide (residues 315-329, RIQRGPGRAFVTIGK (SEQ ID NO. 15), is also presented by class II MHC molecules of both mice (Takahashi et al., J. Exp. Med. 171:571, 1990 incorporated herein by reference) and humans (Clerici et al., Nature 339:383, 1989, incorporated herein by reference) to CD4+ helper T cells.

Based on the above, various P18 peptides may be selected as peptide antigens for use within the co-stimulation methods of the invention. Often, P18 peptide antigens are ten residue peptides which represent the highly immunogenic regions of the V3 loop of various HIV isolates, for example having the amino acid sequences: RGPGRAFVTI (IlIb isolate) (SEQ ID NO. 16), IGPGRAFYTT (MN isolate) (SEQ ID NO. 17), IGPGRAFYAT (SC isolate) (SEQ ID NO. 18), KGPGRVIYAT (RF isolate) (SEQ ID NO. 19), IGPGRAFHTT (SF2 isolate) (SEQ ID NO. 20), IGPGRTLYAR (NYS isolate) (SEQ ID NO. 21), LGPGRVWYTT (CDC4 isolate) (SEQ ID NO. 22), IGPGRAFRTR (WMJ2 isolate) (SEQ ID NO. 23), of the HIV-l gp160 envelope protein. Additional peptides based on the P18 prototype are also operable within the invention, including the peptides RIQRGPGRAFVTIGK (isolate IIIB) (SEQ ID NO. 24), RIHIGPGRAFYTTKN(isolate MN) (SEQ ID NO. 25), SITKGPGRVIYATGQ (isolate RF) (SEQ ID NO. 26), SIHIGPGPAFYATGD (isolate SC) (SEQ ID NO. 27), SLSIGPGRAFRTREI (isolate WMJ-2) (SEQ ID NO. 28), SISIGPGRAFFATTD (isolate Z321) (SEQ ID NO. 29), SIYIGPGRAFHTTGR (isolate SF2) (SEQ ID NO. 30), GIAIGPGRTLYAREK (isolate NY5) (SEQ ID NO. 31), RVTIJGPGRVWYTTGE (isolate CDC4) (SEQ ID NO. 32), SIRIGPGKVFTAKGG(isolate Z3) (SEQ ID NO. 33), GIHFGPGQALYTTG I (isolate MAL) (SEQ ID NO. 34), STPIGLGQM.JYTTRG (isolate Z6) (SEQ ID NO. 35), STPIGLGQALYTTRI (isolate JYl) (SEQ ID NO. 36), and RTPTGLGQSLYTTRS (isolate E-LI) (SEQ ID NO. 37).

Additional HIV peptide antigens (designated by source protein/amino acid sequence/and position) for use within the invention include P21 (gpl20/QIDSKLREQFGNNK/410-429) (SEQ ID NO. 38); (gpl20/GSDTITLPCRIKQFINMWQE/644-658) (SEQ ID NO. 39); P41 (gp41/NYTSLIHSLIEESQN/664-678) (SEQ ID NO. 40); P42 (gp41/EQELLELDKWASLWN/787-801) (SEQ ID NO. 41); P47 (gp41/RIVELLGRRGWEALK/172-196) (SEQ ID NO. 42); (pol(rt)/IETVPVKLKPGMDGPKVKQWPLTEE/325-349) (SEQ ID NO. 43); (pol(rt)/AIFQSSMTKILEPFRKQNPDIVIYQ/342-366) (SEQ ID NO. 44); (pol(rt)/NPDIVIYQYMDDLYVGSDLEIGQHR/359-383) (SEQ ID NO. 45); (pol(rt)/DLEIGQHRTKIEELRQHLLRWGLTT/461-485) (SEQ ID NO. 46); (pol(rt)/PLTEEAELELAENREILKEPVHGVY/495-519) (SEQ ID NO. 47); and (pol(rt)/EIQKQGQGQWTYQIYQEPFKNLKTG/265-279) (SEQ ID NO. 48) (Sequence numbers for gpl20 and gp41 are from Ratner et al., Nature 313:277-284, 1985, and sequence numbers for po1 and gag proteins from Sciliciano et al., Cell 54:561, 1988, and Walker et al., Proc. Natl. Acad. Sci. USA 86:9514, each incorporated herein by reference).

Yet additional peptides and proteins comprising epitopes from HIV proteins, including pol, gag, vpr, vif, rev and env proteins, are useful within the methods of the invention (see, e.g., Palker et al., Proc. Natl. Acad. Sci. USA 85:1932-1936, 1988; Palker et al., J. Immunol. 142:3612-3619, 1989; Hart et al., J. Immunol. 145:2677-2685, 1990 and Hart et al., 1991, supra, each incorporated herein by reference). For example, U.S. Pat. No. 5,756,666 (incorporated herein by reference) discloses various peptides demonstrated to elicit T-cell-mediated immune responses specific to HIV in mammalian subjects. Exemplifying these additional known HIV peptide antigens that are readily incorporated and assessed for operability within the invention, are the following peptides (identified by peptide name, sequence identification, sequence, and position):

```
HIV(B35)-14 (SEQ ID NO.49)
                              NPDIVIYQY
                                    pol 330-338
HIV(B35)ARV2-8 (SEQ ID NO.50)
                              FPVRPQVPL
                                    nef 72-80
HIV(B35)-16 (SEQ ID NO:51)
                              TPPLVKLWY
                                    pol 574-582
HIV(B35)-18 (SEQ ID NO:52)
                              EPIVGAETFY
                                    pol 587-596
HIV(B35)POL-20 (SEQ ID NO:53)
                              SPAIFQSSM
                                    pol 311-319
HIV(B35)ARV2-11 (SEQ ID NO:54)
                              YPLTFGWCF
                                    nef 139-147
HIV(B35)ARV2-25 (SEQ ID NO:55)
                              EPIVGAETF
                                    pol 587-595
HIV(B35)ARV2-4 (SEQ ID NO:56)
                              VPLDKDFRKY
                                    pol 273-282
HIV(B35)ARV2-6 (SEQ ID NO:57)
                              RPQVPLRPMTY
                                    nef 75-85
HIV(B35)ARV2-24 (SEQ ID NO:58)
                              IPLTEEAEL
                                    pol 448-456
HIV(B35)ARV2-33 (SEQ ID NO:59)
                              DPNPQEVVL
                                    env 77-85
HIV(B35)ARV2-36 (SEQ ID NO:60)
                              RPIVSTQLL
                                    env 255-263
HIV(B35)ARV2-38 (SEQ ID NO:61)
                              LPCRIKQII
                                    env 413-421
```

In relation to more detailed aspects of the invention, studies have been conducted testing 9, 10 and 11 residue peptides derived from HIV p18, overlapping or contained within the p 18-I-10 peptide, including specifically both possible 9 residue peptides contained within p18-I-10, and all of these have been found to be less active than p18-I-10. This finding concerning the importance of length in the activity of peptides presented by NBC class I molecules and the identification of a truncation of p18, p18-I-10 (residues 318-327), with 10 to 10²-fold greater potency of T-cell stimulation provides general guidance for selecting other peptide or protein antigens for use within the invention.

Comparable disclosures of useful peptide antigen sequences, e.g., detailing structures of T cell immunogenic proteins, protein fragments, peptides and specific epitopes (including minimal CTL epitope sequences) are provided in the art for other viral antigens that are equally useful within the co-stimulation methods of the invention. In the case of hepatitis B virus (HBV), numerous of this virus' antigenic proteins have been determined and finely mapped to define antigenic domains, fragments, and specific epitopes. In this context, U.S. Pat. Nos. 5,840,303, 5,788,969, 5,780,036, 5,709,995 (each incorporated herein by reference) define a large assemblage of CTL epitopes, including minimal epitopes, of HBV for use as peptide immunogens or incorporation into protein immunogens, conjugates or fusions for coordinate administration with a vector for expressing one or more co-stimulatory molecule(s). Supplementing these disclosures and providing additional description of HBV peptide and protein antigens for use within the invention having T and B cell determinants are U.S. Pat. Nos. 4,599,231 and 4,599,230 (each incorporated herein by reference). In the case of hepatitis C virus (HCV), antigens of this virus are also finely mapped to define antigenic domains, fragments, and specific epitopes for use within the invention (see, e.g., U.S. Pat. No. 5,709,955, inch).

For peptide or protein antigen selection in general, it is notable that both cytotoxic T lymphocytes (CTL) and T helper cells recognize processed antigenic peptides in association with the products of the major histocompatibility complex (NBC) (Schwartz, R. H., Annu. Rev. Immunol. 3:237, 1985; Rosenthal, A. S., Immunol Rev. 40:136, 1978; Benacerraf, B., J. Immunol. 120:1809, 1978; Zinkernagel et al., Adv. Immunol. 27:51, 1979; and Townsend et al., 1989, supra, each incorporated herein by reference). Although naturally processed peptides associate with newly formed MHC class I molecules intracellularly (Germain, supra; and Braciale et al., Immunol. Rev. 98:95, 1987, each incorporated herein by reference), extracellular loading of surface class I molecules by synthetic peptides is commonly used to analyze MHC class I peptide interactions (Townsend et al., Cell 44:959, 1986, incorporated herein by reference). Studies provide substantial evidence that peptides bound to class I are typically approximately nine amino-acids in length (Van Bleek et al., Nature 348:213, 1990; Rotzschke et al., Nature 348:252, 1990; Falk et al., Nature 351:290, 1991; Elliott et al., Nature 351:402, 1991; Madden et al., Nature 353:321, 1991; Jaredetzky et al., Nature 353:326, 1991; and Schumacher et al., Nature 350:703, 1991, each incorporated herein by reference). However, larger peptides are clearly capable of sensitizing targets for class I MHC-restricted lysis. Accordingly, peptide antigens of the invention are often short CTL peptides of 8, 9 or 10 residues in length. However, other peptides and proteins useful within the methods and compositions range exhibit lengths ranging incrementally longer in size, i.e., from 11, 12, 13 or more residues, up to 25-50 residues or longer.

Further guidance in constructing peptide or protein antigens for use within the invention is provided by studies demonstrating that cytotoxic T cell epitopes are recognized by specific, polymorphic HLA Class I or Class II molecules. If only one such CTL epitope, represented by one linear sequence of the peptide, is present in the antigen for vaccine use, then only those individuals with the specific HLA antigen that the peptide uses to be presented to cytotoxic T cells would develop cytotoxic T cells against HIV. However, if multiple CTL epitopes, each capable of being recognized in the context of a distinct HLA Class I or Class II molecule by cytotoxic T cells, are contained in an immunogen (i.e., in a single, contiguous peptide or protein antigen comprising multiple, covalently linked determinants or multiple peptide antigens for coordinate administration with the vector for expressing the co-stimulatory molecule), then individuals with a wide spectrum of HLA-types will generate cytotoxic T cell responses against the target pathogen or cancer cells. Thus, an immunogen capable of inducing cytotoxic T cells in the majority of people in a population, advantageously contains a mixture of peptides, each recognized by a distinct HLA Class I type such that together, the multideterminant peptide or mixture includes epitopes that are immunogenic and recognized by Class I types of molecules that, taken together, are expressed by the majority of individuals in a given population.

The same considerations of MHC restriction that apply to T cytotoxic epitopes also apply to T helper epitopes. That is, recognition of antigens by T helper cells is HLA restricted and for the majority of members of a population cohort to respond to an immunogen and generate a T helper cell response to the immunogen, sufficient T helper cell epitopes will need to be present in order to have available sufficient varieties of T helper epitopes within which each patients' T cells may be able to see processed antigen in the context of their own HLA Class II molecules. To exemplify this aspect of the invention, Table 1 presents a list of T helper cell epitopes of HIV proteins that can be combined in a peptide or formulation of multiple peptides to provide a multideterminate primary signal for T helper cell induction:

TABLE 1
T Cell Helper Epitopes in HIV Proteins

| Peptide | Amino | |
|---|---|---|
| Name | Acid | Protein |
| gp41-1 | 603-614 | gp41 |
| gp41-2 | 609-620 | gp41 |
| gp41-3 | 655-667 | gp41 |
| gp41-5 | 737-749 | gp41 |
| gp41-7 | 584-609 | gp41 |
| gp120-1 | 108-119 | gp120 |
| gp120-2 | 115-126 | gp120 |
| gp120-4 | 296-312 | gp120 |
| gp120-5 | 368-377 | gp120 |
| gp120-6 | 74-85 | gp120 |
| gp120-7 | 233-244 | gp120 |
| p24-1 | 466-473 | p24 |
| p24-2 | 439-446 | p24 |
| p24-3 | 228-235 | p24 |
| p24-4 | 22-29 | p24 |
| p24-11 | 282-301 | p24 |
| pol-1 | 899-913 | pol |
| pol-2 | 923-937 | pol |
| pol-3 | 942-954 | pol |
| pol-4 | 720-730 | pol |
| T1 | 428-443 | gp120 |
| T2 | 112-124 | gp120 |
| TH4 | 834-848 | gp41 |
| p18 | 315-329 | gp120 |

Sequences for the above peptides are from Schrier et al., J. Immunol. 142:1166-1176, 1989, and Clerici et al., Nature 339:383-385, 1989, each incorporated herein by reference.

The peptide or protein antigens employed within the compositions and methods of the invention may be obtained by any of a number of well-known methods in the art; for example, total organic peptide synthesis. In selecting the peptide or peptides to be administered to a subject, it is desirable to provide an epitope which is not present in normal (i.e., non-infected and non-cancerous) cells. For immunization against a virus, it is expected that any of the proteins made by the virus may be useful as target sequences for developing peptide or protein antigens having corresponding sequences (i.e., sequences that across the length of the peptide or protein match a corresponding portion of the full-length sequence of a model antigen), as it is expected that uninfected cells do not express the viral proteins. If a vaccine against a tumor cell is desired, proteins produced by the tumor cell which are not normally made by the host are used as a model or source for producing corresponding peptide or protein antigens.

Identification of proteins which are produced in a tumor cell that are not normally present in the host can be accomplished by several methods, including a comparison by electrophoresis of the total protein profile of the tumor cells and comparing that profile to that of a normal cell of the same tissue. However, it is more convenient to identify mutations in normal cellular proteins that have led to the tumor phenotype. This is accomplished routinely by sequencing of a nucleic acid obtained from a sample of the tumor tissue.

When choosing peptide or protein antigens to synthesize and use within the methods of the invention, the sequence of the peptide or protein may be designed to render the peptide more soluble. Also it is desirable that the peptide sequence be one that is easily synthesized, that is, a sequence that lacks highly reactive side groups. Furthermore, the peptide need not be the minimal peptide that will bind to the MHC protein, as longer sequences will be processed and presented sufficiently to elicit the desired, primary T-cell response.

Both the peptide or protein antigens and vectors for expressing co-stimulatory molecules for use within the invention are provide for formulation of a vaccine or for direct administration in a purified form. The term “purified” as used herein, is intended to refer to a peptide or nucleic acid composition, isolated from naturally associated proteins and other contaminants, wherein the peptide or nucleic acid is purified to a measurable degree relative to its naturally-occurring state, e.g., relative to its purity within a cell extract. In certain embodiments, “purified” will refer to a peptide or polynucleotide composition which has been subjected to fractionation to remove various non-protein components, such as other cell components. Various techniques suitable for use in protein purification are well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques.

In certain embodiments of the invention, purified peptide or protein antigens will be chemically synthesized by solid phase synthesis and purified away from the other products of the chemical reactions, for example by HPLC. Alternatively, the polypeptide may be produced by the overexpression of a DNA sequence included in a vector in a recombinant cell. In this method of producing the peptide, purification may be accomplished by an variety of appropriate technique known in the art.

It is understood that antigenic peptides of the invention may incorporate a single T lymphocyte epitope or multiple epitopes (multideterminant peptides). In addition, it is well known that antigenic determinants are sometimes discontinuous and, therefore, two or more peptide segments of the same antigenic protein may be required for immunoreactivity. All such peptide segments, either continuous or discontinuous, and even including full-length proteins recognized by T lymphocytes are understood to be encompassed by the present invention and to lie within the scope of the present claims. For example, purified polypeptides which include the sequences designated herein as peptide or protein antigens useful within the invention and having a length of less than 100 amino acids, sometimes less than 50 amino acids, more typically less than 25 amino acids, and most often 10 amino acids or less are included within the scope of the present claimed invention.

It is also understood that a peptide or protein antigen of the present invention may be fused, generally by genetic techniques well known to those of skill in the art, to a carrier protein or peptide and that such a protein-peptide or peptide-peptide fusion which exhibits immunoreactivity with T lymphocytes is also encompassed by the present invention. For example, a polypeptide that contains any of the amino acid sequences designated herein as peptide or protein antigens and further including the amino acid sequences of .beta.-galactosidase or glutathione-S-transferase, for example would also be an embodiment of the present invention. It is understood that these and other carrier protein sequences are mentioned by way of example only and that other known carrier protein sequences such as keyhole limpet hemocyanin (KLH), bovine serum albumin (BSA), other albumins such as ovalbumin, mouse serum albumin or rabbit serum albumin or any other suitable protein sequence may also be included as embodiments of the present invention. It is further understood that the use of the term “protein” does not limit the invention to polypeptides or peptides of any particular size. Peptides from as small as several amino acids in length to proteins of any size as well as protein-peptide fusions are encompassed by the present invention, so long as the protein or peptide is capable of providing a primary antigenic signal to activate T lymphocytes.

Within the methods and compositions of the invention, co-stimulatory molecules are also selected for their ability to stimulate T cell activation (e.g., as marked by T cell proliferation and/or increased cytokine production. In addition, co-stimulatory molecules are selected that enhance antigen specific immune responses in the subject when coordinately administered with a peptide or protein antigen of interest. Exemplary co-stimulatory molecules for use within the invention include B7-1, B7-2, B7-3, B7-H, ICAM1, ICAM2, ICAM3, LFA1, LFA2, and LFA3. Any one or combination of multiple of these or additional co-stimulatory molecules can be incorporated within the co-stimulatory vectors of the invention for enhancement of a T lymphocyte immune response.

Structural details and other relevant information are known in the art for each the foregoing co-stimulatory molecules as well as other co-stimulatory molecules not enumerated herein, and are incorporated herein for descriptive purposes to elucidate specific aspects of the invention. In this regard, numerous published resources furnish details describing the structure and function and/or providing the relevant nucleotide and amino acid sequences of co-stimulatory genes and proteins, operable for use within the invention (see, e.g., Parra et al., J. Immunol. 158:637-642, 1997; Wingren et al., Crit Rev. Immunol. 15:235-253, 1995; Swallow et al., Immunity 11:423-432, 1999; Bleijs et al., Eur. J. Immunol. 29:2248-2258, 1999; Kotovuori et al., J. Immunol. 162:6613-6620, 1999; Salomon et al., J. Immunol. 161:5138-5142, 1998; Carpenito et al., Scand. J. Immunol. 45:248-254, 1997). For example, specific references are available and incorporated herein that provide relevant structural (e.g., amino acid or nucleotide sequence) information concerning ICAM-1 (Staunton et al., Cell 52:925-933, 1988); LFA-3 (Wallner et al., J. Exp. Med. 166:923-932, 1987); B7-H (Dong et al., Nat. Med. 5:1365-1369, 1999); B7-1 (Selvakumar et al., Immunogenetics 36:175-181, 1992; U.S. Pat. Nos. 5,861,310 and 5,942,607), B7-2 (Azuma et al., Nature 366:76-79, 1993; U.S. Pat. Nos. 5,861,310 and 5,942,607) and B7-3 U.S. Pat. Nos. 5,861,310 and 5,942,607). Each of these references are incorporated herein for ease of description, particularly with respect to their disclosures pertaining to native structures of co-stimulatory genes and their encoded proteins.

In related aspects of the invention, the peptide or protein antigens and co-stimulatory proteins for vaccine use include naturally occurring peptide and protein variants, e.g., naturally occurring allelic variants and mutant proteins, as well as synthetic, e.g., chemically or recombinantly engineered variants, including biologically active peptide fragments, and analogs of the peptide or protein antigens or co-stimulatory proteins. As used herein, protein or peptide “analogs” is meant to include a protein or peptide incorporating one or more amino acid substitutions, insertions, rearrangements or deletions as compared to a native amino acid sequence of the subject protein or peptide (e.g., a native B7-1, B7-2, or B7-3 amino acid sequence as described in U.S. Pat. Nos. 5,861,310 and 5,942,607, or a native amino acid sequence of a T cell epitope or antigen of a virus or other pathogen as described herein). Protein and peptide analogs thus modified exhibit co-stimulatory or primary immune inducing activity comparable to that of native protein or peptide (e.g., activity that is at least 50%, typically at least 75% or greater compared to activity of native protein or peptide as determined by a suitable in vitro assay or in vivo assay).

As used herein, the term “biologically active protein or peptide fragments or analogs” refers to fragments or analogs of a co-stimulatory molecules or peptide or protein antigens which do not encompass the full length, or depart from the native amino acid sequence, of the subject co-stimulatory molecule or analog, but which nevertheless maintain activity for primary T cell stimulation or co-stimulation comparable to the native peptide or protein in an appropriate assay system, e.g., a cytokine production or CTL cytolysis assay. These biologically active protein or peptide fragments or analogs are all intended to be encompassed by the generic terms “co-stimulatory molecule” and “peptide or protein antigen”, as well as by the specific names for co-stimulatory molecules (e.g., the specific namnes B7-l, B7-2, B7-3, B7H, ICAM1, ICAM2, LFA1 and LFA2) and peptide or protein antigens (e.g., P18 and subtype variants thereof) used herein.

For purposes of the present invention, useful analogs of peptide or protein antigens and co-stimulatory molecules also includes derivatives or synthetic variants, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Where a peptide or protein antigen or co-stimulatory protein is derivatized by amino acid substitution, amino acids are generally replaced by other amino acids having similar, conservatively related chemical properties such as hydrophobicity, hydrophilicity, electronegativity, bulky side chains and the like. Residue positions which are not identical to a native protein or peptide sequence are thus replaced by amino acids having similar chemical properties, such as charge or polarity, which changes are not likely to substantially effect the properties of the antigen or co-stimulatory molecule. These and other minor alterations substantially maintain the immunoidentity (e.g., recognition by one or more monoclonal antibodies that recognize the native antigen or co-stimlatory molecule) and other biological activities of the native antigen or co-stimlatory molecule.

In this context, the term “conservative amino acid substitution” refers to the general interchangeability of amino acid residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between glycine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

Analogs of peptide or protein antigens and co-stimulatory molecules for use within the invention also include proteins or peptide fragments incorporating stereoisomers (e.g., D-amino acids) of the twenty conventional amino acids, or unnatural amino acids such as α,α-disubstituted amino acids, N-alkyl amino acids, lactic acid. These and other unconventional amino acids may be substituted or inserted within native amino acid sequences of peptide or protein antigens and co-stimulatory proteins of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). Moreover, amino acids may be modified by glycosylation, phosphorylation and the like.

For purposes of the present invention, analogs of peptide or protein antigens and co-stimulatory proteins also include single or multiple substitutions, deletions and/or additions of carbohydrate, lipid and/or other proteinaceous moieties that occur naturally or artificially as structural components of the peptide or protein antigens and co-stimulatory proteins, or are bound or otherwise associated with the peptide or protein.

Analogs of peptide or protein antigens and co-stimulatory proteins may be readily constructed, e.g., using peptide synthetic techniques well known in the art such as solid phase peptide synthesis (Merrifleld synthesis) and the like, or by recombinant DNA techniques well known in the art. Techniques for making substitution mutations at predetermined sites in DNA include for example M13 mutagenesis. Manipulation of DNA sequences to produce substitutional, insertional, or deletional variants are conveniently described elsewhere such as Sambrook et al., 1989, supra. In accordance with these and related teachings, defined mutations can be introduced into a native peptide or protein antigen or co-stimulatory protein to generate analogs of interest by a variety of conventional techniques, e.g., site-directed mutagenesis of a CDNA copy of the peptide or protein antigen or co-stimulatory protein. This can be achieved through and intermediate of single-stranded form, such as using the MUTA-gen® kit of Bio-Rad Laboratories (Richmond, CA), or a method using the double-stranded plasmid directly as a template such as the Chameleon® mutagenesis kit of Strategene (La Jolla, CA), or by the polymerase chain reaction employing either an oligonucleotide primer or a template which contains the mutation(s) of interest. A mutated subfragment can then be assembled into a complete analog-encoding cDNA. A variety of other mutagenesis techniques are known and can be routinely adapted for use in producing the mutations of interest to generate analogs of peptide or protein antigens and co-stimulatory proteins for use within the invention.

To facilitate production and use of useful analogs of peptide or protein antigens and co-stimulatory proteins within the invention, reference can be made to molecular phylogenetic principals to characterize conserved and divergent structural and functional elements between related peptide or protein antigens and co-stimulatory proteins. In this regard, available studies referenced provide data for analysis of co-stimulatory protein structure-function on a molecular level. These studies allow for detailed sequence comparisons to identify conserved and divergent structural elements among related co-stimulatory molecules and antigenic peptides, yielding maps of conserved structural and functional domains, sequence elements and constrained, isolated residues within these peptides and proteins. Each of these conserved and divergent protein domains, structural motifs, sequence elements and constrained residues facilitate practice of the invention by providing useful targets for incorporation of conservative or divergent sequence elements within analogs of peptide or protein antigens and co-stimulatory proteins to confer desired structural and/or functional changes.

In this context, existing sequence alignments may be analyzed, or conventional sequence alignment methods may be employed to yield sequence comparisons for analysis, to identify corresponding protein regions and amino acid positions between native peptide or protein antigens and co-stimulatory proteins and a related or homologous protein bearing a structural element of interest for incorporation within analogs of the peptide or protein antigens and co-stimulatory proteins. Typically, one or more amino acid residues marking a structural element of interest in a different reference protein or peptide is incorporated within the protein or peptide analog. For example, a cDNA encoding B7-1 may be recombinantly modified at one or more corresponding amino acid position(s) of a B7-2, B7-3 or B7H reference protein (i.e., corresponding positions that match or span a similar aligned sequence element according to accepted alignment methods to residues marking the structural element of interest in the reference protein) to encode an amino acid deletion, substitution, or insertion that alters corresponding residue(s) in the B7-1 protein to generate an analog having an analogous structural and/or functional element as the reference protein. Within this rational design method for constructing analogs of peptide or protein antigens and co-stimulatory proteins, the wild-type identity of residue(s) at amino acid positions corresponding to a structural element of interest in the target protein for mutagenesis may be altered to the same, or a conservatively related, residue identity as the corresponding amino acid residue(s) in the reference protein. However, it is also possible to alter the native amino acid residues non-conservatively with respect to the corresponding reference protein residue(s) (e.g., by using any other amino acid to disrupt or impair the identity and function of the wild-type residue(s)).

Analogs of peptide or protein antigens and co-stimulatory proteins typically show substantial sequence identity to a corresponding reference amino acid sequence of a parental or homologous peptide or protein antigen or co-stimulatory protein as described herein. The terms “substantial sequence identity” mean that two amino acid sequences, when optimally aligned, such as by the programs GAP or BESTFIT using default gap penalties, share at least 80 percent sequence identity, often at least 90-95 percent or greater sequence identity. “Percentage amino acid identity” or “percentage amino acid sequence identity” refers to a comparison of the amino acids of two polypeptides which, when optimally aligned, have approximately the designated percentage of the same amino acids. Sequence comparisons are generally made to a reference sequence over a comparison window of at least 5, typically at least nine or ten, more typically at least 20 nucleotide positions, and frequently over a window of at least 25-50 nucleotides, wherein the percentage of sequence identity is calculated by comparing the reference sequence to the polynucleotide sequence, which may include deletions or additions which total 20 percent or less of the reference sequence over the window of comparison. The reference sequence may be a subset of a larger sequence, for example, as a segment of a native peptide or protein antigen and co-stimulatory protein sequence describe herein. Optimal alignment of sequences for aligning a comparison window may be conducted according to the local homology algorithm of Smith and Waterman (1981) Adv. Anpl. Math. 2:482, by the homology alignment algorithm of Needleman and Wunsch (1970) J. Mol. Biol. 48:443, by the search for similarity method of Pearson and Lipman (1988) Proc. Natl. Acad. Sci. USA 85:2444, or by computerized implementations of these algorithms (GAP, BESTFIT, FASTA, and TFASTA in the Wisconsin Genetics Software Package Release 7.0, Genetics Computer Group, 575 Science Dr., Madison, Wis.).

By aligning a protein optimally with a native sequence of a peptide or protein antigen or co-stimulatory protein, and by using appropriate assays, e.g., immunoassays, cytokine assays, or cytolysis assays, to determine a selected biological activity, one can readily identify operable analogs of peptide or protein antigens and co-stimulatory proteins for use within the methods and compositions of the invention. For example, homologous co-stimulatory proteins from different vertebrate species, for example from different mammalian or primate species, are typically specifically immunoreactive with antibodies raised to a corresponding murine or human B7-1, B7-2, B7-3, B7H, ICAM1, ICAM2, LFA1 or LFA2 molecule disclosed herein. Likewise, nucleic acids encoding functional B7-1, B7-2, B7-3, B7H, ICAM1, ICAM2, LFA1 and LFA2 analogs and fragments of the invention will typically selectively hybridize to a corresponding nucleic acid sequence encoding a native murine or human B7-1, B7-2, B7-3, B7H, ICAM1, ICAM2, LFA1 or LFA2 molecule under accepted, moderate or high stringency hybridization conditions (see, e.g., Sambrook et al., 1989 Molecular Cloning: A Laboratory Manual, Cold Spring Harbor Laboratories, Cold Spring Harbor, N.Y., incorporated herein by reference). Less stringent hybridization conditions may also be selected.

Generally, analogs of peptide or protein antigens and co-stimulatory proteins for use within the invention will hybridize to corresponding, native or homologous, nucleic acid sequences under stringent conditions selected to be about 5° C. lower than the thermal melting point (Tm) for the subject sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents and the extent of base mismatching, the combination of parameters is more important than the absolute measure of any one. Thus, the phrase “selectively hybridizing to” refers to a nucleic acid probe that hybridizes, duplexes or binds preferentially to a particular target DNA or RNA sequence when the target sequence is present in a preparation of total cellular DNA or RNA. “Complementary” or “target” nucleic acid sequence refers to a nucleic acid sequence which selectively hybridizes to a nucleic acid probe. For discussions of nucleic acid probe design and annealing conditions, see, for example, Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed., Greene Publishing and Wiley-Interscience, N.Y. (1987), each of which is incorporated herein by reference.

Thus, the present invention includes analogs of peptide or protein antigens and co-stimulatory proteins encoded by nucleotide sequences that share substantial sequence identity or homology to corresponding sequences of native or homologous peptide or protein antigens and co-stimulatory proteins disclosed herein. For substantial sequence identity or homology the analog-encoding polynucleotide comprises a sequence that has at least 80 percent sequence identity, preferably at least 90 percent sequence identity, and more preferably at least 95 percent sequence identity compared to a corresponding sequence of the native peptide or protein. The comparison is made to a reference sequence over a comparison window of at least 20 nucleotide positions, frequently over a window of at least 25-50 nucleotides, wherein the percentage of sequence identity is calculated by comparing the reference sequence to the polynucleotide sequence, which may include deletions or additions which total 20 percent or less of the reference sequence over the window of comparison. The reference sequence may be a subset of a larger sequence, for example, as a segment of a murine or human B7-1, B7-2, B7-3, B7H, ICAM1, ICAM2, LFA1 or LFA2CAP-3 sequence described herein. Optimal alignment of sequences for aligning a comparison window may be conducted according to the local homology algorithm of Smith and Waterman, Adv. Appl. Math. 2:482, 1981 by the homology alignment algorithm of Needleman and Wunsch, J. Mol. Biol. 48:443, 1970, by the search for similarity method of Pearson and Lipman, Proc. Natl. Acad. Sci. USA 85:2444, 1988 or by computerized implementations of these algorithms (GAP, BESTFIT, FASTA, and TFASTA in the Wisconsin Genetics Software Package Release 7.0, Genetics Computer Group, 575 Science Dr., Madison, Wis.).

Various methods for isolating nucleic acid molecules encoding peptide or protein antigens and co-stimulatory molecules are available to identify and clone new antigens or co-stimulatory proteins, and/or to generate analogs of existing peptide or protein antigens and co-stimulatory proteins. For example, DNA encoding a co-stimulatory protein can be isolated from a genomic or cDNA library using labeled oligonucleotide probes having sequences complementary to the sequences of known co-stimulatory proteins described herein. Full-length probes may be used, or oligonucleotide probes also may be generated according to known methods, and such probes can be used directly in hybridization assays to isolate DNA encoding new co-stimulatory molecules. Alternatively, probes can be designed for use in amplification techniques such as PCR (Mullis, et al., U.S. Pat. Nos. 4,683,195 and 4,683,202, incorporated herein by reference), and DNA encoding peptide or protein antigens and co-stimulatory proteins may be isolated by using methods such as PCR. Nucleic acid probes may be DNA or RNA fragments. DNA fragments can be prepared, for example, by digesting plasmid DNA, or by use of PCR, or synthesized by either the phosphoramidite method described by Beaucage and Carruthers, Tetrahedron Lett. 22:1859-1862, 1981 or by the triester method according to Matteucci et al., J. Am. Chem. Soc. 103:3 185, 1981, both incorporated herein by reference. A double stranded fragment may then be obtained, if desired, by annealing the chemically synthesized single strands together under appropriate conditions or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

To prepare a cDNA library, mRNA is isolated from tissue such as human placenta which expresses CAP-3 protein. cDNA is prepared from the MRNA and ligated into a recombinant vector. The vector is transfected into a recombinant host for propagation, screening and cloning. Methods for making and screening cDNA libraries are well known. See Gubler and Hoffmnan Gene 25:263-269, 1983 and Sambrook, et al., supra.

For a genomic library, the DNA is extracted from tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro, as described in Sambrook, et al. Recombinant phage are analyzed by plaque hybridization as described in, for example, Benton and Davis, Science, 196:180-182, 1977. Colony hybridization is carried out as generally described in, for example, Grunstein, et al., Proc. Natl. Acad. Sci. USA., 72:3961-3965, 1975.

DNA encoding a peptide or protein antigen or co-stimulatory protein of interest is identified in either cDNA or genomic libraries by its ability to hybridize with nucleic acid probes, for example on Southern blots, and these DNA regions are isolated by standard methods familiar to those of skill in the art. See Sambrook, et al. Various methods of amplifying target sequences, such as the polymerase chain reaction, can also be used to prepare nucleic acids encoding peptide or protein antigens and co-stimulatory proteins. PCR technology is used to amplify such nucleic acid sequences directly from mRNA, from CDNA, and from genomic libraries or cDNA libraries. The isolated sequences encoding CAP-3 protein may also be used as templates for PCR amplification.

In PCR techniques, oligonucleotide primers complementary to the two 3′ borders of the DNA region to be amplified are synthesized. The polymerase chain reaction is then carried out using the two primers. See PCR Protocols: A Guide to Methods and Applications Innis, M., Gelfand, D., Sninsky, J. and White, T., eds., Academic Press, San Diego (1990). Primers can be selected to amplify the entire regions encoding a full-length co-stimulatory protein or to amplify smaller DNA segments as desired.

PCR can be used in a variety of protocols to isolate cDNAs encoding peptide or protein antigens and co-stimulatory proteins. In these protocols, appropriate primers and probes for amplifying DNA encoding the peptide or protein antigens and co-stimulatory proteins are generated from analysis of the DNA sequences listed herein. Once such regions are PCR-amplified, they can be sequenced and oligonucleotide probes can be prepared from sequence obtained. These probes can then be used to isolate DNAs encoding peptide or protein antigens and co-stimulatory proteins.

Oligonucleotides for use as probes are typically chemically synthesized according to the solid phase phosphoramidite triester method first described by Beaucage and Carruthers, Tetrahedron Lett. 22:1859-1862, 1981, using an automated synthesizer, e.g., as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom. 255:137-149, 1983. The sequence of the synthetic oligonucleotide can be verified using the chemical degradation method of Maxam and Gilbert, Meth. Enzymol. 65:499-560, 1984. Other methods known to those of skill in the art may also be used to isolate DNA molecules encoding peptide or protein antigens and co-stimulatory proteins. See Sambrook, et al. for a description of other techniques for the isolation of DNA encoding specific protein molecules.

Non-viral vectors for delivery of co-stimulatory molecules within the invention optionally comprise RNA or DNA vectors. In one aspect of the invention, the vector comprises a “naked DNA” vector that incorporates the polynucleotide encoding the co-stimulatory molecule operably linked to regulatory elements necessary for expression of the co-stimulatory molecule in eukaryotic cells. Direct injection of naked DNA expression vectors into vertebrate tissues via intraperitoneal, intravenous and intramuscular routes has been shown to result in the uptake of DNA and long-term expression of the protein encoded by the DNA (Acsadi et al., Nature 352:815-818, 1991; Benvenisty et al.; Wolff et al., Science 247:1464-1468, 1990; 1990, Nabel et al., Science 249:1286-1288, 1990; Fynan et al., Proc. Natl. Acad. Sci. USA 90:11478-11482, 1990; W090/11092 (Oct. 4, 1990); and U.S. Pat. No. 5,866,553 issued Feb. 2, 1999, each incorporated herein by reference).

The non-viral vectors of the invention comprise nucleic acids that contain essential regulatory elements such that upon introduction into a living vertebrate cell they are able to direct the cellular machinery to produce translation products from the co-stimulatory factor-encoding polynucleotide. There are many embodiments of the invention which those skilled in the art can appreciate from the specification. For example, different transcriptional promoters, terminators, carrier vectors or specific gene sequences may be used. In one embodiment, the polynucleotide encoding the co-stimulatory molecule is cloned into a nucleotide expression vector, typically a DNA expression vector.

The vector typically contains a promoter for RNA polymerase transcription, and a transcriptional terminator at the end of the co-stimulatory molecule-encoding sequence. In addition, to assist in preparation of the pharmaceutical, an antibiotic resistance marker expressed in E. coli may be included in the expression vector. Neomycin resistance genes or any other pharmaceutically acceptable antibiotic resistance marker may be used. Further, to aid in the high level production of the pharmaceutical by fermentation in prokaryotic organisms, it is advantageous for the vector to contain an origin of replication and be of high copy number. A variety of commercially available prokaryotic cloning vectors provide these benefits and are well known and readily available to the artisan. In preparing expression vectors for use within the invention it is generally desirable to remove non-essential DNA sequences.

Numerous expression systems are available for expression of DNA encoding co-stimulatory molecules of the invention. The expression of natural or synthetic nucleic acids encoding these molecules, such as B7, is typically achieved by operably linking the DNA to a promoter (which is either constitutive or inducible) within an expression vector. Typically, the expression vector is a DNA molecule, linear or circular, that comprises a segment encoding a co-stimulatory molecule, operably linked to

additional segments that provide for its transcription. As noted above, such additional segments include promoter and terminator sequences. DNA vectors for use within the invention also may include one or more origins of replication, one or more selectable markers, an enhancer, a polyadenylation signal, etc. Expression vectors generally are derived from plasmid or viral DNA, and can contain elements of both. The term “operably linked” indicates that the segments are arranged so that they function in concert for their intended purposes, for example, transcription initiates in the promoter and proceeds through the coding segment to the terminator (see, e.g., Sambrook et al., Molecular Cloning: A Laboratory Manual, Cold Spring Harbor Laboratories, Cold Spring Harbor, N.Y., 1989, incorporated herein by reference).

Mammalian expression vectors for use in carrying out the present invention include a promoter capable of directing the transcription of a cloned gene or cDNA. Either viral promoters or cellular promoters can be used. Viral promoters include the immediate early cytomegalovirus (CMV) promoter (Boshart et al., Cell 41:521-530, 1985, incorporated herein by reference) and the SV40 promoter (Subramani et al., Mol. Cell. Biol. 1:854-864, 1981, incorporated herein by reference). Cellular promoters include the mouse metallothionein-1 promoter (Palmiter et al., U.S. Pat. No. 4,579,821, incorporated herein by reference), a mouse VI) promoter (Bergman et al., Proc. Natl. Acad. Sci. USA 81:7041-7045, 1983; and Grant et al., Nuc. Acids Res. 15:5496, 1987, each incorporated herein by reference), a mouse VH promoter (Loh et al., Cell 33:85-93, 1983, incorporated herein by reference), and the major late promoter from Adenovirus 2 (Kaufinan and Sharp, Mol. Cell. Biol. 2:1304-13199, 1982, incorporated herein by reference).

A variety of approaches are available for introducing functional genetic material into cells via non-viral vectors for ex vivo and in vivo delivery of co-stimulatory molecules within the methods of the invention (for review, see, e.g., Anderson, Science 256:808-813, 1992, incorporated herein by reference). Operable methods within the invention include delivery of a transgene linked to a heterologous promoter-enhancer element via liposomes coupled to ligand-specific, cation-based transport systems (Friedmann, Science 244:1275-1281, 1989; Brigham et al., Am. J. Med. Sci. 298:278-281, 1989; Nabel et al., Science 249:1285-1288, 1990; Hazinski et al., Am. J. Resp. Cell Molec. Biol. 4:206-209, 1991; Wang and Huang, Proc. Natl. Acad. Sci. USA 84:7851-7855, 1987; and Wu and Wu, J. Biol. Chem. 263:14621-14624, 1988, each incorporated herein by reference). Alternatively, the co-stimulatory molecule may be delivered by the use of naked DNA expression vectors (Nabel et al., supra; Wolff et al., supra, each incorporated herein by reference). Direct injection of transgenes into tissue produces localized expression suitable for achieving co-stimulation by coordinate administration of the vector with an T cell antigenic peptide (Rosenfeld, 1992, supra); Rosenfeld et al., 1991, supra; Brigham et al., 1989, supra; Nabel et al., supra; and Hazinski et al., 1991, supra). The Brigham et al. group (Am. J. Med. Sci. 298:278-281, 1989, incorporated herein by reference) have reported in vivo transfection of lungs of mice following either intravenous or intratracheal administration of a DNA liposome complex.

Yet additional guidance for refining methods of production and delivery of non-viral vectors encoding co-stimulatory molecules is provided in PCT/US90/01515 (incorporated herein by reference), directed to methods for delivering a gene coding for a pharmaceutical or immunogenic polypeptide to the interior of vertebrate cells in vivo. PCT/US90/05993 (incorporated herein by reference) is directed to a method for obtaining expression of a transgene in mammalian lung cells following either intravenous or intratracheal injection of an expression construct. PCT 89/02469 and PCT 90/06997 (each incorporated herein by reference) are directed to ex vivo gene therapy, i.e., which is useful within the invention for expressing a transgene encoding a co-stimulatory molecule in cells (e.g., lymphocytes), tissues and organs that can be taken out of the body. PCT 89/12109 (incorporated herein by reference) is likewise directed to ex vivo gene therapy. PCT 90/12878 (incorporated herein by reference) describes an enhancer element which provides a high level of expression both in transformed cell lines and in transgenic mice using ex vivo transfection. PCT/US92/08806 (incorporated herein by reference) is directed to particle-mediated transformation of mammalian cells.

In more detailed embodiments of the invention, a transgene vector comprising nucleotide sequence encoding a T cell co-stimulatory molecule (e.g., B7-1, B7-2, B7-3, B7H, ICAM1, ICAM2, LFA1 or LFA2) is administered to a target cell or tissue by any of several techniques, depending at least in part on the nature of the target cell or tissue, accessibility of the target cell or tissue, and the intended result of therapy. In “ex vivo” vector administration, target cells or tissues are transfected ex vivo followed by introduction of the transformed cells or tissues into a suitable organ in the host mammal. Ex vivo therapy and vaccination within the methods of the invention include transfection of cells or tissues in vitro with either naked DNA or DNA liposome conjugates, followed by introduction of the transfected cells or tissues into a host subject. The criteria for a suitable target cell, tissue or organ include that the target cell, tissue or organ is accessible, can be manipulated in vitro, and is susceptible to genetic modification methods. Further, it should be possible to reimplant the genetically modified cells, tissues or organs into the organism in a functional and stable form. Exemplary of a target in this context is the mammalian bone marrow. If only differentiated, replicating cells are transfected, the newly introduced gene function will be lost as those cells mature and die, which can be advantageous for treating acute diseases and/or symptoms as the above methods generally involve integration of new genetic material into the cell genome and thus constitute permanent changes. Notably, the foregoing ex vivo methods can be employed for various diagnostic purposes, for example to detect T cell responses in subjects to evaluate the status or presence of pathogenic infections or cancer.

The ease of producing and purifying DNA constructs that direct expression of co-stimulatory molecules for use within the invention compares favorably with that of traditional protein purification, which facilitates the generation of vaccine compositions. In this regard, multiple constructs collectively encoding multiple co-stimulatory molecules, e.g., B7-1 and B7-2, may be readily produced for simultaneous or sequential administration within the methods of the invention. Because protein expression may be maintained for an extended period of time following DNA injection, the persistence of B-and T-cell memory may be enhanced, thereby engendering long-lived humoral and cell-mediated immunity.

In each of the foregoing embodiments of the invention, the peptide or protein antigen and vector for expressing the co-stimulatory molecule are delivered in a manner consistent with conventional methodologies associated with management of the pathogenic infection or cancer condition for which treatment or prevention is sought. In accordance with the disclosure herein, an immunogenically effective amount of the antigen and vector is administered to a subject in need of such treatment for a time and under conditions sufficient to prevent, inhibit, and/or ameliorate the infection or cancer condition being treated. The term “subject” as used herein means any vertebrate patient, typically a mammalian patient, to which the compositions of the invention may be beneficially administered. Subjects specifically intended for treatment with the compositions and methodologies of the present invention include humans, as well as non-human primates, sheep, horses, cattle, goats, pigs, dogs, cats, rats, mice and avian subjects. Alternate subjects for treatment using the methods of the invention include cells, cell explants, tissues and organs originating from these subjects.

Subjects for treatment according to the invention include patients at high risk for developing cancer or pathogenic infection, as well as patients presenting with existing cancer conditions or infections. To identify subject patients for prophylaxis or treatment, or prevention of recurrence of disease, according to the methods of the invention, accepted screening methods are employed to determine risk factors associated with cancer or pathogenic infection, or to determine the status of an existing cancer disorder or infection identified in a subject. These screening methods include, for example, conventional work-ups to determine familial status for a particular cancer disorder known to have a heritable component. Toward this end, nucleotide probes can be routinely employed to identify individuals carrying genetic markers associated with a particular cancer disorder of interest. In addition, a wide variety of immunological methods are known in the art that are useful to identify markers for specific cancer conditions and pathogenic infections. For example, various ELISA immunoassay methods are available and well known in the art that employ monoclonal antibody probes to detect antigens associated with cancer cells or viral or non-viral pathogens. Such screening may be implemented as indicated by known patient symptomatology, age factors, related risk factors, etc. These methods allow the clinician to routinely select patients in need of treatment according to the methods of the invention. In accordance with these methods and principles, the methods of the invention may be implemented as an independent prophylaxis or treatment program or as a follow-up, adjunct or coordinate treatment regimen to other treatments, for example surgery, drug therapy, vaccination, immunotherapy, cell, tissue, or organ transplants, and the like.

As noted above, the methods of the invention involve administering a immunogenically effective amount of a peptide or protein antigen and a non-viral vector for expressing a co-stimulatory molecule. The antigen and vector are formulated, separately or as a “chimeric vaccine” formulation, with a pharmaceutically acceptable carrier and administered in an amount sufficient to elicit a T lymphocyte-mediated immune response. According to the methods of the invention, the peptide or protein antigen and vector for expressing the co-stimulatory molecule may be administered to subjects by a variety of administration modes, including by intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen and vector may be administered ex-vivo by direct exposure to cells, tissues or organs originating from a subject, optionally in a biologically suitable, liquid or solid carrier.

In certain embodiments of the invention, the peptide or protein antigen and vector for expressing the co-stimulatory molecule are delivered to a common or adjacent target site in the subject, for example to a specific target tissue or cell population in which the vaccine formulation is intended to elicit an immune response. Typically, when the peptide or protein antigen and vector are administered separately, they are delivered to the same or closely proximate site(s), for example to a single target tissue or to adjacent sites that are structurally or fluidly connected with one another (e.g., to allow direct exposure of the same cells, e.g., fluid flow transfer, dissipation or diffusion through a fluid or extracellular matrix of both vaccine agents). Thus, a shared target site for delivery of antigen and vector can be a common surface (e.g., a mucosal, basal or lunenal surface) of a particular target tissue or cell population, or an extracellular space, lumen, cavity, or structure that borders, surrounds or infiltrates the target tissue or cell population.

For prophylactic and treatment purposes, the peptide or protein antigen and vector encoding the co-stimulatory molecule may be administered to the subject separately or together, in a single bolus delivery, via continuous delivery (e.g., continuous intravenous or transdermal delivery) over an extended time period, or in a repeated administration protocol (e.g., on an hourly, daily or weekly basis). The various dosages and delivery protocols contemplated for administration of peptide and vector, in simultaneous or sequential combination, are immunogenically effective to inhibit the occurrence or alleviate one or more symptoms of the target infection or cancer disorder in the subject. An “immunogenically effective amount” of the antigen and vector thus refers to an amount that is, in combination, effective, at dosages and for periods of time necessary, to elicit a specific T lymphocyte mediated immune response. This response can be determined by conventional assays for T-cell activation, including but not limited to assays to detect specific cytokine activation and/or cytolytic activity.

In more detailed aspects of the invention, the amount of peptide or protein antigen and of the vector encoding the co-stimulatory molecule, determined based on separate or combined dosages of antigen and vector, are immunogenically effective to achieve a desired antiviral, antibacterial, or cancer inhibitory effect in the subject. In specific embodiments, an immunogenically effective amount of antigenic peptide and co-stimulatory vector, depending on the selected mode, frequency and duration of administration, will effectively prevent viral, bacterial or parasitic infection or cancer, or will inhibit progression of an infectious or cancerous condition in the subject. Alternatively or in addition to these effects, an immunogenically effective dosage of the antigen and vector, which may include repeated doses within an ongoing prophylaxis or treatment regimen, will alleviate one or more symptoms or detectable conditions associated with an infectious or cancerous disorder. This includes any detectable symptom or condition amenable to prophylaxis and/or treatment with the vaccines of the invention, for example symptoms or conditions associated with infection by human immunodeficiency virus (HIV), hepatitis B virus (HBV), herpes simplex virus (HSV) or human papilloma virus (HPV), or one or more conditions associated with breast cancer, cervical cancer, prostate cancer, colon cancer, melanoma and other cancerous conditions.

For prophylactic and therapeutic use, peptide or protein antigens and vectors encoding co-stimulatory proteins of the invention are typically formulated with a pharmaceutically acceptable carrier. As used herein, “pharmaceutically acceptable carrier” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound.

Peptide or protein antigens may be administered to the subject in the form of a peptide solution per se or a combination of a peptide with an appropriate auxiliary agent using an injector. Alternatively, the peptide or protein antigen may be percutaneously administered through mucous membrane by, for instance, spraying the solution. The unit dose of the peptide typically ranges from about 0.01 mg to 100 mg, more typically between about 100 μg to about 5 mg, which may be administered, one time or repeatedly, to a patient. Moreover, it is often more effective to simultaneously administer a plurality of antigenic peptides, or a formulation of a multideterminate peptide (e.g., a conjugate of a PCLUS peptide and a P18 peptide) by the foregoing administering method to provide multideterminant vaccination.

Examples of auxiliary agents which can be formulated with or conjugated to peptide or protein antigens and/or vectors for expressing co-stimulatory molecules to enhance their immunogenicity for use within the invention include bacterial cell components such as BCG bacterial cell components, imnunostimulating complex (ISCOM), extracted from the tree bark called QuillA (Morein et al., Nature 308:457, 1984; and Nature 344:873, 1990, each incorporated herein by reference), QS-21, a saponin-type auxiliary agent (J. Immunol. 148:1438, 1992, incorporated herein by reference), liposomes (J. Immunol. 148:1585, 1992; incorporated herein by reference), aluminum hydroxide (alum), bovine serum albumin (BSA), tetanus toxoid (TT) (Green et al., Cell 28:477, 1982; and Palker et al., Proc. Natl. Acad. Sci. USA 84:2479, 1987, each incorporated herein by reference) and keyhole limpet hemocyanin (KLH) (J. Virol. 65:489, 1991, incorporated herein by reference). In addition, Deres et al., Nature 342:561, 1989 (incorporated herein by reference), have shown that conjugation of a fatty acid tripalmitoyl-S-glycerylcysteinyl-seryl-serine moiety to synthetic peptides can promote synthetic peptide processing and presentation in the context of MHC Class I molecules. That the foregoing methods facilitate induction of immune response such as a CTL response in the living body is also detailed in the aforementioned references, and in Science, 255:333, 1992 (incorporated herein by reference).

In more detailed aspects of the invention, peptide or protein antigens are formulated with or conjugated to one or more additional antigenic determinants. Exemplary of these aspects of the invention, multideterminant peptides (comprising a plurality of epitopes selected from CTL, T helper and B cell epitopes) for eliciting specific immune responses against HIV to provide a primary signal within the co-stimulatory methods of the invention are disclosed by Berzofsky and colleagues (U.S. Pat. No. 5,939,074; U.S. Pat. No. 5,976,541; U.S. Pat. No. 5,030,449; U.S. Pat. No. 5,081,226; U.S. Pat. No. 5,932,218; U.S. patent application Ser. No. 08/060988, filed May, 14, 1993, corresponding to EP 0 710 572 B1; WO 94/26785; and U.S. patent application Ser. No. 09/455,076, each incorporated herein by reference). Within specific embodiments of the invention, the peptide or protein antigen comprises a multideterminant cluster peptide of a HIV envelope protein, for example PCLUS 1, PCLUS 2, PCLUS 3, PCLUS 4, PCLUS 5, PCLUS 6, and PCLUS 6.1, as disclosed in the above-incorporated references. Also available for use within the invention are peptide or protein antigens that comprise a multideterminant cluster peptide of a HIV envelope protein and a second peptide, for example a P18 HIV peptide, that contains a T- or B-cell epitope. Thus, for example, a peptide or protein antigen for use within the invention, for use against HIV or any other selected pathogen, may comprise a first peptide sequence having an epitope derived from a target antigen that is capable of eliciting a-helper T cell response, a second peptide sequence having an epitope capable of eliciting a cytotoxic T cell response, and a third peptide sequence having an epitope capable of eliciting a high titer neutralizing antibody response to the same viral antigen.

In preparing pharmaceutical compositions of the present invention, it may be desirable to modify the peptide or protein antigen, non-viral vector, or co-stimulatory molecule encoded by the vector, or to combine or conjugate the peptide or co-stimulatory protein with other agents, to alter pharmacokinetics and biodistribution. For a general discussion of pharmacokinetics, see Remington's Pharmaceutical Sciences, supra, Chapters 37-39. A number of methods for altering pharmacokinetics and biodistribution are known to persons of ordinary skill in the art. Examples of such methods include protection of the proteins, protein complexes and polynucleotides in vesicles composed of other proteins, lipids (for example, liposomes), carbohydrates, or synthetic polymers. For example, the vaccine agents of the invention can be incorporated into liposomes in order to enhance pharmacokinetics and biodistribution characteristics. A variety of methods are available for preparing liposomes, as described in, e.g., Szoka et al., Ann. Rev. Biophys. Bioeng. 2:467, 1980; and U.S. Pat. Nos. 4,235,871, 4,501,728 and 4,837,028, each incorporated herein by reference. For use with liposome delivery, peptide or protein antigens and vectors for expressing co-stimulatory molecules are typically entrapped within the liposome, or lipid vesicle, or is bound to the outside of the vesicle.

Within certain embodiments of the invention, peptide or protein antigens and vectors encoding co-stimulatory molecules are associated with liposomes, such as lecithin liposomes or other liposomes known in the art, as a DNA-liposome mixture, or the DNA may be associated with an adjuvant known in the art to boost immune responses, such as a protein or other carrier. Additional agents which assist in the cellular uptake of DNA, such as, but not limited to, calcium ions, viral proteins and other transfection facilitating agents and methods may also be used to advantage (Wigler et al., Cell 14:725, 1978; Corsaro and Pearson, Somatic Cell Genetics 7:603, 1981; Graham and Van der Eb, Virology 52:456, 1973; Neumann et al., EMBO J. 1:841-845, 1982; and Hawley-Nelson et al., Focus 15:73-79, 1993, each incorporated herein by reference).

Several strategies have been devised to increase the effectiveness of liposome-mediated drug delivery by targeting liposomes to specific tissues and specific cell types. Examples of liposome formulations that can be employed within the methods and compositions of the invention include DOTAP™ (Boehringer-Mannheim), Tfx^{™}-50, Transfectam®, ProFection™ (Promega, Madison, Wis.), and LipofectAmin™, Lipofectin®, LipofectAce™ (GibcoBRL, Gaithersburg, Md.). The use of specific cationic lipids can confer specific advantages for in vivo delivery of complexes. For example, intravenous injection of nucleic acid complexed to DOTAP-containing liposomes or ethyl-phosphatidylcholine (E-PC) lipid carriers can target transgene expression primarily to the lung. Furthermore, DOTAP, as well as L-PE and cholesterol ester β-alanine (CEBA) are fully metabolized by cells, whereas DOTMA cannot be fully metabolized by cells. Therefore, DOTAP, E-PC, and L-PE, but not DOTMA, are suitable for repeated injection into mammalian hosts. Additionally, using a lipid carrier comprising a cationic lipid and a second lipid, particularly cholesterol or DOPE can maximize transgene expression in vivo. Also, mixing a steroid, such as cholesterol, instead of DOPE, with DOTAP, DOTMA, or DDAB, substantially increases transgene expression in vivo. In solution, the lipids form vesicles that associate with the nucleic acid and facilitate its transfer into cells by fusion of the vesicles with cell membranes or by endocytosis. Liposome formulation, including those containing a cationic lipid, have been shown to be safe and well tolerated in human patients (Treat et al., J. Natl. Cancer Instit. 82:1706-1710, 1990).

A variety of additional methods may be employed to enhance delivery of nucleic acids encoding co-stimulatory molecules into subjects to induce the expression of these proteins. For example, a specialized means of delivery for introducing the vectors is by coating them on gold microprojectiles and introducing the particles into the subject, e.g., intradermally or subcutaneously. Jet injectors or “gene guns” are also well known in the art and useful to transfect skin, muscle, fat, and mammary tissues of living animals. These and other methods for introducing nucleic acids into subjects are reviewed by Donnelly et al., (The Immunologist 2:20, 1994, incorporated herein by reference).

In vivo expression of transgenes encoding co-stimulatory molecules can also be obtained by injection of transgenes directly into a specific tissue, such as direct intratracheal, intramuscular or intraarterial injection of naked DNA or of DNA-cationic liposome complexes, or by ex vivo transfection of host cells, with subsequent reinfusion. The expression generally is limited to one tissue, typically the tissue that is injected (for example muscle); liver or lung where intravenous injection is used; lung where intratracheal injection is used, brain where intrathecal injection is used, and heart where injection via a catheter is used. Particular cells and tissues can be targeted, depending upon the route of administration and the site of administration. For example, a tissue which is closest to the site of injection in the direction of blood flow can be transfected in the absence of any specific targeting. Additionally, if desired, the lipid carriers may be modified to direct the complexes to particular types of cells using site-directing molecules. Thus, antibodies or ligands for particular receptors or other cell surface proteins may be employed, with a target cell associated with a particular surface protein.

Vaccine formulations comprising, separately or in admixed compositions, a peptide or protein antigen and a vector for expressing a co-stimulatory molecule alternatively contain as pharmaceutically acceptable carriers substances required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, and generally 10-95%, more typically 25% -75% of active ingredient(s).

Vaccine compositions of the invention can be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

In certain embodiments of the invention, peptide or protein antigens and/or vectors encoding co-stimulatory proteins are administered in a time release formulation, for example in a composition which includes a slow release polymer, or by depot injection. The active protein or peptide can be prepared with carriers that will protect against rapid release, for example a controlled release vehicle such as implants, transdermal patches, or microencapsulated delivery system. Prolonged delivery of vaccine agents of the invention can be brought about by inclusion in the composition of agents delaying absorption, for example, aluminum monostearate hydrogels and gelatin. When controlled release formulations are desired, controlled release binders suitable for use in accordance with the invention include any biocompatible controlled-release material which is inert to the active ingredient and which is capable of incorporating the peptide or protein antigens and/or vectors encoding the co-stimulatory proteins. Numerous such materials are known in the art. Useful controlled-release binders are materials which are metabolized slowly under physiological conditions following their subcutaneous or intramuscular injection in mammals (i.e., in the presence of bodily fluids which exist there). Appropriate binders include but are not limited to biocompatible polymers and copolymers previously used in the art in sustained release formulations. Such biocompatible compounds are non-toxic and inert to surrounding tissues, e.g., following subcutaneous or intramuscular injection, and do not trigger significant adverse effects such as immune response, inflammation, or the like. They are metabolized into metabolic products which are also biocompatible and easily eliminated from the body.

For example, a polymeric matrix derived from copolymeric and homopolymeric polyesters having hydrolysable ester linkages may be used. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (i.e., L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof. Many methods for preparing such formulations are generally known to those skilled in the art (see, e.g., Sustained and Controlled Release Drug Delivery Systems, J. R. Robinson, ed., Marcel Dekker, Inc., N.Y., 1978, incorporated herein by reference).

In more detailed embodiments, useful vaccine formulations include controlled-release compositions such as are known in the art for the administration of leuprolide (trade name: Lupron.RTM.), e.g., microcapsules (U.S. Pat. Nos. 4,652,441 and 4,917,893, each incorporated herein by reference), injectable formulations (U.S. Pat. No. 4,849,228, incorporated herein by reference), lactic acid-glycolic acid copolymers useful in making microcapsules or injectable formulations (U.S. Pat. Nos. 4,677,191 and 4,728,721, each incorporated herein by reference), and sustained-release compositions for water-soluble peptides (U.S. Pat. No. 4,675,189, incorporated herein by reference). A long-term sustained release implant also may be used. These can be readily constructed to deliver therapeutic levels of peptide or protein antigens and vectors encoding co-stimulatory proteins for at least 10-20 days, often at least 30 days, up to 60 days or longer. Long-term sustained release implants are well known to those of ordinary skill in the art and can incorporate some of the absorption delaying components described above. Such implants can be particularly useful by placing the implant near or directly within the target tissue or cell population, thereby affecting localized, high-doses of vaccine agents at one or more target sites.

In alternate embodiments, peptide or protein antigens and vectors encoding co-stimulatory proteins are orally or rectally administered for treatment or prophylaxis of cancer or viral disease, for example, with an inert diluent or an assimilable edible carrier. Examples of methods for mucosal delivery, e.g., rectal mucosal delivery, of vaccine agents for incorporation within the methods of the invention to elicit specific CTL responses in a subject are disclosed in International Publication No. WO 99/12563 (incorporated herein by reference). In other detailed embodiments peptide or protein antigens and vectors encoding co-stimulatory proteins may be formulated for oral or rectal delivery enclosed in a hard or soft shell gelatin capsule, compressed into tablets, or incorporated directly into the subject's diet. For oral therapeutic administration, vaccine agents may be incorporated with excipients and used in the form of ingestable tablets, buccal tablets, troches, capsules, elixirs, suspensions, syrups, wafers, and the like. The percentage (e.g., by weight or by volume) of individual vaccine agents in these compositions and other preparations described herein may, of course, be varied.

For oral or rectal administration, vaccine agents of the invention can be worked into tablets or other solid forms by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such as magnesium stearate, sodium lauryl sulfate, “Carbowax” or polyethylene glycol. Of course, taste-improving substances can be added in the case of oral administration forms. Solid delivery vehicles may contain CAP-3 protein or peptide in mixture with fillers, such as lactose, saccharose, mannitol, starches, such as potato starch or amylopectin, cellulose derivatives or highly dispersed silicic acids. In soft-gelatin capsules, the active substance is dissolved or suspended in suitable liquids, such as vegetable oils or liquid polyethylene glycols. As further forms, one can use plug capsules, e.g., of hard gelatin, as well as dosed soft-gelatin capsules comprising a softener or plasticizer, e.g. glycerin.

Alternatively, liquid dosage forms for delivering vaccine agents to mucosal surfaces include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms would also include minerals and other materials to make them compatible with the type of injection or delivery system chosen.

The prophylactic and therapeutic compositions of the invention typically must be sterile and stable under all conditions of manufacture, storage and use. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In certain embodiments of the invention, vaccine agents (i.e., peptide or protein antigen, vector for expressing a co-stimulatory molecule, or one or more single or multiple determinant peptide(s) formulated in combination with a vector encoding a co-stimulatory molecule) is administered by topical delivery to a mucosal surface of the patient, for example via intranasal or intrapulmonary delivery in the form of an aerosol spray or powder. According to one aspect of the invention, the vaccine agents are delivered in an intranasally effective amount, typically in a selected volume of administered spray or powder, to achieve a desired therapeutic result. In related aspects of the invention, novel pharmaceutical compositions are provided for intranasal delivery that incorporate the vaccine agent(s) in a powder or aqueous formulation for intranasal delivery. Intranasal administration allows self-administration of treatment by patients, provided that sufficient safeguards are in place to control and monitor dosing and side effects. Nasal administration also overcomes certain drawbacks of other administration forms, such as injections, that are painful and expose the patient to possible infections and may present drug bioavailability problems.

Compositions according to the present invention may thus be administered from solution in the form of nasal drops or may be dispensed as a spray by a variety of methods known to those skilled in the art. Systems for intranasally dispensing liquids as a spray are well known. In one embodiment, metered doses of vaccine agent(s) are delivered by means of a specially constructed mechanical pump valve (U.S. Pat. No. 4,511,069, incorporated herein by reference). This hand-held delivery device is uniquely nonvented so that sterility of the solution in the aerosol container is maintained indefinitely. Certain nasal spray solutions comprise one or more vaccine agents in a liquid carrier that optionally includes a nonionic surfactant for enhancing absorption of the drug and one or more buffers or other additives to minimize nasal irritation. In some embodiments, the nasal spray solution further comprises a propellant. The pH of the nasal spray solution is typically between pH 6.8 and 7.2.

For aerosol administration, the pharmaceutical compositions containing peptide or protein antigens and/or vectors encoding co-stimulatory proteins are typically supplied in finely divided form along with a surfactant and propellant as pharmaceutically acceptable carriers (See, e.g., U.S. Pat. No. 5,902,789, incorporated herein by reference). The surfactant must be nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides, can be employed. A carrier also can be included, as desired, as with, for example, lecithin for intranasal delivery. Alternative means of intranasal vaccine administration are provided by the use of ion exchange resins or adsorbent resin powders as carriers (see, e.g., U.S. Pat. No. 5,942,242, incorporated herein by reference).

In more detailed aspects of the invention, vaccine agents, most commonly peptide or protein antigens, are stabilized to extend effective half-life following delivery to the subject, particularly for extending metabolic persistence in an active state within an extracellular compartment (e.g., in the bloodstream, at a mucosal surface, or within a connective tissue compartment or fluid-filled body cavity). For this purpose, vaccine agents may be modified by chemical means, e.g., chemical conjugation, N-terminal capping, PEGylation, or recombinant means, e.g., site-directed mutagenesis or construction of fusion proteins, or formulated with various stabilizing agents or carriers. Thus stabilized, CAP-3 administered as above retains anti-inflammatory and autoimmune inhibitory activity for an extended period at the intended target site.

Numerous reports in the literature describe the potential advantages of pegylated polypeptides, which include their increased resistance to proteolytic degradation, increased plasma half-life, increased solubility and decreased antigenicity and immunogenicity (Nucci, et al., Advanced Drug Deliver Reviews 6:133-155, 1991; Lu et al., Int. J. Peptide Protein Res. 43:127-138, 1994, each incorporated herein by reference). A number of proteins, including L-asparaginase, strepto-kinase, insulin, and interleukin-2 have been conjugated to a poly(ethyleneglycol) (PEG) and evaluated for their altered biochemical properties as therapeutics (see, e.g., Ho, et al., Drug Metabolism and Disposition 14:349-352, 1986; Abuchowski et al., Prep. Biochem. 9:205-211, 1979; and Rajagopaian et al., J. Clin. Invest. 75:413-419, 1985, each incorporated herein by reference). Although the in vitro biological activities of pegylated proteins may be decreased, this loss in activity is usually offset by the increased in vivo half-life in the bloodstream (Nucci, et al., Advanced Drug Deliver Reviews 6:133-155, 1991, incorporated herein by reference).

Several procedures have been reported for the attachment of PEG to proteins and peptides and their subsequent purification (Abuchowski et al., J. Biol. Chem. 252:3582-3586, 1977; Beauchamp et al., Anal. Biochem. 131:25-33, 1983, each incorporated herein by reference). Lu et al., (Int. J. Peptide Protein Res. 43:127-138, 1994, incorporated herein by reference) describe various technical considerations and compare PEGylation procedures for proteins versus peptides (see also, Katre et al., Proc. Natl. Acad. Sci. USA 84:1487-1491, 1987; Becker et al., Makromol. Chem. Rapid Commun. 3:217-223, 1982; Mutter et al., Makromol. Chem. Rapid Commun. 13:151-157, 1992; Merrifield, R. B., J. Am. Chem. Soc. 85:2149-2154, 1993; Lu et al., Peptide Res. 6:142-146, 1993; Lee et al., Bioconjugate Chem. 10:973-981, 1999, Nucci et al., Adv. Drug Deliv. Rev. 6:133-151, 1991; Francis et al., J. Drug Targeting 3:321-340, 1996; Zalipsky, S., Bioconjugate Chem. 6:150-165, 1995; Clark et al., J. Biol. Chem. 271:21969-21977, 1996; Pettit et al., J. Biol. Chem. 272:2312-2318, 1997; Delgado et al., Br. J. Cancer 73:175-182, 1996; Benhar et al., Bioconjugate Chem. 5:321-326, 1994; Benhar et al., J. Biol. Chem. 269:13398-13404, 1994; Wang et al., Cancer Res. 53:4588-4594, 1993; Kinstler et al., Pharm. Res. 13:996-1002, 1996, Filpula et al., Exp. Opin. Ther. Patents 9:231-245, 1999; Pelegrin et al., Hum. Gene Ther. 9:2165-2175, 1998, each incorporated herein by reference). Following these and other teachings in the art, the conjugation of antigenic peptides and polypeptides with poly(ethyleneglycol) polymers, is readily undertaken with the expected result of prolonging circulating life and/or reducing immunogenicity while maintaining an acceptable level of activity of the PEGylated derivative.

Amine-reactive PEG polymers for use within the invention include SC-PEG with molecular masses of 2000, 5000, 10000, 12000, and 20 000; U-PEG-10000; NHS-PEG-3400-biotin; T-PEG-5000; T-PEG-12000; and TPC-PEG-5000. Chemical conjugation chemistries for these polymers have been published (see, e.g., Zalipsky, S., Bioconjugate Chem. 6:150-165, 1995; Greenwald et al., Bioconjusate Chem. 7:638-641, 1996; Martinez et al., Macromol. Chem. Phys. 198:2489-2498, 1997; Hermanson, G. T., Bioconijugate Techniques, pp. 605-618, 1996; Whitlow et al., Protein Eng. 6:989-995, 1993; Habeeb, A. F. S. A., Anal. Biochem. 14:328-336, 1966; Zalipsky et al., Poly(ethyleneglycol) Chemistry and Biological Applications, pp. 318-341, 1997; Harlow et al., Antibodies: a Laboratory Manual, pp. 553-612, Cold Spring harbor Laboratory, Plainview, N.Y., 1988; Milenic et al., Cancer Res. 51:6363-6371, 1991; Friguet et al., J. Immunol. Methods 77:305-319, 1985, each incorporated herein by reference). While phosphate buffers are commonly employed in these protocols, the choice of borate buffers may beneficially influence the PEGylation reaction rates and resulting products.

PEGylation of peptides and proteins for stabilized delivery within the methods of the invention may be achieved by modification of carboxyl sites (e.g., aspartic acid or glutamic acid groups in addition to the carboxyl terminus) of the protein or peptide. The utility of PEG-hydrazide in selective modification of carbodiimide-activated protein carboxyl groups under acidic conditions has been described (Zalipsky, S., Bioconjugate Chem. 6:150-165, 1995; Zalipsky et al., Poly(ethyleneglycol) Chemistry and Biological Applications, pp. 318-341, American Chemical Society, Washington, DC, 1997, incorporated herein by reference). Alternatively, bifunctional PEG modification of CAP-3 can be employed. In some procedures, charged amino acid residues, including lysine, aspartic acid, and glutamic acid, have a marked tendency to be solvent accessible on protein surfaces. Conjugation to carboxylic acid groups of proteins is a less frequently explored approach for production of protein bioconjugates. However, the hydrazide/EDC chemistry described by Zalipsky and colleagues (Zalipsky, S., Bioconjugate Chem. 6:150-165, 1995; Zalipsky et al., Poly(ethyleneglycol) Chemistry and Biological Applications, pp. 318-341, American Chemical Society, Washington, DC, 1997, each incorporated herein by reference) offers a practical method of linking PEG polymers to protein carboxylic sites. For example, this alternate conjugation chemistry has been shown to be superior to amine linkages for PEGylation of brain-derived neurotrophic factor (BDNF) while retaining biological activity (Wu et al., Proc. Natl. Acad. Sci. U.S.A. 96:254-259, 1999, incorporated herein by reference). Maeda and colleagues have also found carboxyl-targeted PEGylation to be the preferred approach for bilirubin oxidase conjugations (Maeda et al., Poly(ethylene alycol) Chemistry. Biotechnical and Biomedical Applications, J. M. Harris, Ed., pp. 153-169, Plenum Press, N.Y., 1992, incorporated herein by reference).

In addition to PEGylation, vaccine agents of the invention can be modified to enhance circulating half-life by shielding the agents via conjugation to other known protecting or stabilizing compounds, or by the creation of fusion proteins comprising active proteins or peptides with other active or inactive proteins or peptides, or example immunoglobulin chains (see, e.g., U.S. Pat. Nos. 5,750,375, 5,843,725 5,567,584 and 6,018,026, each incorporated herein by reference). These modifications will decrease the degradation, sequestration or clearance of vaccine agents in vivo and result in a longer half-life of the protein, for example in the circulatory system, lung or synovium.

In additional aspects of the invention, peptide or protein antigens and vectors encoding co-stimulatory proteins are administered according to the foregoing methods in a coordinate therapy protocol with one or more additional immune modulatory agents, for example anti-inflammatory or immune inhibiting agents. Thus, in various embodiments peptide or protein antigens and vectors encoding co-stimulatory proteins are administered simultaneously or sequentially with administration of one or more supplemental immune modulatory agents. These secondary or adjunctive therapeutic agents may comprise a glucocorticoid or non-steroidal anti-inflammatory drug (NSAID). Glucocorticoids useful within this aspect of the invention include short-acting glucocorticoids (e.g., cortisone and hydrocortisone), intermediate-acting glucocorticoids (e.g., prednisone, prednisolone, peprednisone, methylprednisolone and triamcinolone), and long-acting glucocorticoids (betamethasone, dexamethasone and paramethasone). NSAID's useful within the invention include, aspirin, salicylates, naproxen, indomethacin, piroxicam, oxaprozin, phenylbutazone, ibuprofen, flurbiprofen, fenoprophen, and ketoprofen).

Other secondary therapeutic agents for coordinate administration within, or in conjunction with, vaccine formulations of the invention include cytotoxic drugs, T-cell specific inhibitors, antisera, replacement immune globulins, and monoclonal antibodies, (for example, the monoclonal antibody, muromonab-CD3 (Orothoclone OKT3) which blocks CD3/T-cell receptor interactions essential for signal transduction). Lymphocyte immune globulin (Atgam) is useful in allotransplantation because it markedly suppresses T-lymphocyte mediated immune responses. Specific examples of immunosuppressive drugs useful in conjunction with vaccine therapy according to the invention include azathioprine, cyclophosphamide, and cyclosporine. A new immunosuppressive drug is FK-506 which suppresses alloantigen or T-cell mitogen-induced lymphocyte proliferation and significantly prolongs survival of organ and skin grafts is particularly effective. Cytotoxic drugs for coordinate administration with peptide or protein antigens and vectors encoding co-stimulatory proteins include drugs that inhibit B-cell and T-cell responses and which are often used to treat autoimmune disorders (e.g., azathioprine, azathioprine sodium, chloramucil, cyclophosphamide, methotrexate, methotrexate sodium, and clyclosprorine).

In certain alternative additional aspects of the invention, peptide or protein antigens may be deliverable for use within the invention using recombinant live vaccines, for example using vaccinia viruses and BCG bacteria. More specifically, if a DNA coding for an antigenic peptide as described above is incorporated into a gene coding for a recombinant antigen protein expressed in a recombinant live vaccine, the peptide sequence may be expressed as part of the antigenic protein and then presented by an HLA class I antigen through processing thereof within the cells to thus induce CTL which can recognize it. A method for expressing foreign genes in BCG bacterial cells is detailed in International Publication No. W088/06626 (incorporated herein by reference). Recombinant live vaccines derived from BCG bacteria are detailed in J. Exp. Med. 178:197, 1993 (incorporated herein by reference). The dose and the administration method may be determined or selected in conformity to those for the usual vaccination and BCG vaccines. The ability to induce immune responses such as CTL in vivo using such methods is disclosed in, for example, Nature 332:728, 1988; and Nature 351:479, 1991 (each incorporated herein by reference).

In addition to using recombinant live vaccines, peptide or protein antigens may also be amenable to delivery via a non-viral, DNA or RNA vector. This general method of vaccination has been shown to elicit specific immune responses in vertebrate subjects in vivo (Fynan et al., Proc Natl. Acad Sci USA 90:11478-11482, 1990; and U.S. Pat. No. 5,866,553, each incorporated herein by reference). One exemplary method involves the use of naked DNA encoding the antigen, as disclosed, e.g., in WO 90/11092 (Oct. 4, 1990). The efficacy of DNA vaccination to elicit a CTL response, e.g., to protect against subsequent viral challenge, is typically achieved by immunization with non-replicating plasmid DNA encoding one or more of the above mentioned viral proteins. This is advantageous within the co-stimulatory methods of the invention, since no infectious agent is involved, no assembly of virus particles is required, and determinant selection is permitted.

Thus, within certain aspects of the invention non-viral vectors encoding a peptide or protein antigen of interest are administered to the subject coordinately with administration of a non-viral vector for expressing the co-stimulatory molecule. The peptide or protein antigen and co-stimulatory molecule may be encoded by the same vector, with separate or common regulatory elements directing their expression. The vector construct encoding the peptide or protein antigen is capable of being expressed upon direct introduction into animal tissues to induce expression of the encoded peptide within the tissues of the subject. Where the peptide is one that does not occur in that animal except during infections, such as proteins associated with viral infection, the immune system of the animal is activated to launch a protective response. Because these exogenous proteins are produced by cells of the host animal, they are processed and presented by the major histocompatibility complex (MHC). This recognition is analogous to that which occurs upon actual infection with the related organism. This approach to vaccination is applicable to tumors as well as infectious agents, since the CD8+ CTL response is important for immunological intervention in both pathophysiological processes.

Yet additional methods for administration of peptide or protein antigens within the methods of the invention involve delivery of antigen-coated cells, for example peptide-pulsed dendritic cells, to the subject. As described in U.S. Patent Application 08/472,298, filed Jun. 7, 1995 by Berzofsky et al., and corresponding International Publication No. WO 94/21287 (each incorporated herein by reference), the use of coated cells may facilitate presentation of peptide or protein antigens and stimulation of an antigen-specific immune response, thereby enhancing co-stimulation of T lymphocyte activation. Exemplary of these immunoprophylactic and immunotherapeutic methods are those which comprise a method for eliciting tumor-specific CD8+ cytotoxic T lymphocytes in a human or other vertebrate. Within these methods autologous or syngeneic lymphoid or myeloid cells, typically antigen presenting dendritic cells, are coated with a peptide or protein antigen by incubating the cells with the peptide in vitro. The cells are then irradiated, typically with between 1,000 and 3,300 rad gamma irradiation, and then injected or otherwise administered to the subject. At the same time or in a prior or subsequent, coordinate administration, the vector encoding the co-stimulatory molecule is delivered to the subject.

Determination of effective dosages for administration of peptide or protein antigens and vectors for expressing co-stimulatory molecules within the invention is typically based on animal model studies followed up by human clinical trials, and is guided by determining effective dosages and administration protocols that significantly reduce the occurrence or severity of the target infection or cancerous disorder in model subjects. Alternatively, effective dosages can be determined using in vitro models (e.g., cytolytic assays). Using such models, only ordinary calculations and adjustments are typically required to determine an appropriate concentration and dose to administer an effective (e.g., intranasally effective, transdermally effective, intravenously effective, or intramuscularly effective) amount of the peptide and vector to a subject to elicit a desired immune response.

The actual dosage of peptide and vector will of course vary according to factors such as the disease state, age, and weight of the individual, and the ability of the peptide and vector to initiate and co-stimulate a desired T-cell immune response in the individual. Dosage regimens may be adjusted to provide an optimum therapeutic response. A therapeutically effective amount is also one in which any toxic or detrimental side effects of the peptide or protein antigen/co-stimulator therapy is outweighed by therapeutically beneficial effects. A non-limiting range for an immunogenically effective amount of peptide or protein antigen and vector is 0.01 μg/kg-10 mg/kg, and in more specific embodiments between about 0.01 and 5 mg/kg. These values are expressed for the antigen and vector separately, whereas immunological efficacy is determined based on the combined amounts of both antigen and vector delivered to the subject. In determining optimal dosages, a broad incremental range of doses is compared for immunogenicity in order to optimize concentrations for use. Typically, separate dosages of 10, 50, 100, 200 or more μg of the peptide or protein antigen and vector encoding the co-stimulatory molecule DNA are efficacious in man.

Dosages within the foregoing ranges can be achieved by single or multiple administrations, including, e.g., multiple administrations per day, daily or weekly administrations. Per administration, it is desirable to administer at least one microgram each of the peptide or protein antigen and vector, often between 10 μg and 5.0 mg, to an average human subject. It is to be further noted that for each particular subject, specific dosage regimens should be evaluated and adjusted over time according to the individual need and professional judgment of the person administering or supervising the administration of the vaccine compositions.

Dosages of peptide or protein antigen and vector encoding the co-stimulatory molecule may be varied by the attending clinician to maintain a desired concentration at the target site. For example, if an intravenous mode of delivery is selected, local concentrations of the peptide or protein antigen and vector in the bloodstream at the target tissue, e.g., within or surrounding a tumor mass, may be between about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, e.g., intradermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

In evaluating delivery and efficacy of peptide or protein antigens and co-stimulatory molecules within the methods of the invention, levels of peptides and proteins within a tissue or body fluid sample of the subject or in an in vitro sample can be determined, for example, by means of a variety of different immunoassay procedures. Antibodies, both polyclonal and monoclonal, can be produced to peptide or protein antigens and co-stimulatory proteins according to general procedures set forth in Harlow and Lane, Antibodies: A Laboratory Manual, Cold Spring Harbor, N.Y., 1988, incorporated herein by reference. Antibodies raised to these vaccine agents can be selected to be specifically immunoreactive with the peptide or protein of interest and not with other proteins, for example other co-stimulatory factors. A variety of immunoassay formats may be used to select antibodies specifically immunoreactive with a particular protein. For example, solid-phase ELISA immunoassays are routinely used to select monoclonal antibodies specifically immunoreactive with a protein. For a description of immunoassay formats and conditions that can be used to determine specific immunoreactivity see, e.g., Harlow and Lane, supra; Basic and Clinical Immunology, 7th Edition, D. Stites and A. Terr, eds., 1991; Enzyme Immunoassay, E. T. Maggio, ed., CRC Press, Boca Raton, Fla., 1980; and “Practice and Theory of Enzyme Immunoassays” P. Tijssen, Laboratory Techniques in Biochemistry and Molecular Biology, Elsevier Science Publishers B.V., Amsterdam, 1985; each of which is incorporated herein by reference.

To further evaluate and optimize the methods of the invention it may also be desirable to evaluate expression of mRNA encoding peptide or protein antigens and co-stimulatory proteins. MRNA expression can be detected by various procedures involving nucleic acid hybridization. A variety of nucleic acid hybridization formats are known to those skilled in the art. For example, common formats include sandwich assays and competition or displacement assays. Hybridization techniques are generally described in “Nucleic Acid Hybridization, A Practical Approach” Ed. Hames, B. D. and Higgins, S. J., IRL Press, 1985; Gall and Pardue, Proc. Natl. Acad. Sci. U.S.A. 63:378-383, 1969; and John et al., Nature 223:582-587, 1969; and in Sambrook et al., 1989, supra, each incorporated herein by reference. Hybridization techniques can be also used in methods such as restriction fragment length polymorphism (RFLP) analysis to detect the presence of genetic alterations in nucleic acids encoding CAP-3 (see Sambrook et al., 1989, supra).

The following examples are offered to illustrate, but no to limit the claimed invention.

### EXAMPLES

The following examples demonstrate the efficacy of a novel strategy of the invention in which peptide vaccination is combined with coadministration of a DNA vector expressing a co-stimulatory molecule, exemplified the full length B7-1 protein. These examples show that intradermal vaccination of C57BL/6 mice with a DNA vector carrying the murine B7.1 insert, in combination with administration of an E7 peptide antigen, yields a high-level cytolytic response of lymphocytes generated specifically directed against E7 peptide-pulsed target cells. From these and additional, related studies, the co-vaccination methods of the invention have been refined for improved efficacy, e.g., by multiple studies using different peptide antigen and vector doses. The observed CTL responses elicited by the co-stimulatory vaccine formulations and methods of the invention were shown to be dose dependent. From these and related experiments, it is demonstrated that vaccine preparations and methods featuring a peptide immunogen admixed or coordinately administered with a DNA construct expressing a co-stimulatory molecule can be administered to vertebrate subjects to elicit an enhanced T cell-mediated immune response.

### Example I

### Coordinate Administration of HPV E7 Peptide Antigen and a DNA Vector Encoding B7-1 Elicits an Enhance CTL Response In Vivo

The present example documents use of a non-viral, DNA plasmid vector containing the open reading frame (ORF) of the murine B7. 1 co-stimulatory protein coordinately administered with an antigenic peptide, HPV E7(49-57), to enhance a T lymphocyte-mediated immune response directed toward the peptide. In particular, subcutaneous injection of the B7-1 DNA vector prior to the injection of the E7 peptide significantly enhanced the E7-specific CTL response when injection of the B7-1 DNA and E7 peptide directed to the same target site in the subject.

Preparation of Vaccine

A peptide antigen formulation for use within the present example was prepared in an emulsion by a two-syringe system containing 1:1 incomplete Freund's Adjuvant with 100 μg HPV16 E7 peptide (49-57), 20 μg PCLUS-6.1 helper epitope (see, U.S. Pat. No. 5,939,074; U.S. Pat. No. 5,976,541; U.S. Pat. No. 5,030,449; U.S. Pat. No. 5,081,226; U.S. Pat. No. 5,932,218; U.S. patent application Ser. No. 08/060988, filed May 14, 1993, corresponding to EP 0 710 572 Bi; WO 94/26785; and

U.S. patent application Ser. No. 09/455,076, each incorporated herein by reference), 50,000 U IL-2, 10X PBS and double distilled water. The B7-1 expressing DNA plasmid (400 μg) was prepared in 100 μl water.

Immunization of Animal Subjects

8-12 week-old C57BL/6 mice were immunized (in groups of 3) intradermally at the base of the tail with peptide emulsion followed by intradermal injection of B7-1-encoding DNA vector, as describe above. Injections of peptide antigen and of the vector encoding the co-stimulatory molecule were made either at a common site (same side of tail base) or at remote sites, respectively (different sides of tail base) in different test animals.

Cytotoxic Lymphocyte Assay

Procedures to measure CTL activity are well known and are described elsewhere (see, e.g. Chen et al., Cell 71:1093-1102, 1992). In brief, spleens from immunized mice were collected 14 days after vaccination. Spleen cells (3×10⁶/well) were stimulated in 24-well plates with irradiated naive C57BL/6 (3×10⁶/well) spleen cells pulsed for 2 hours with 5 μM of E7 peptide. Rat T-stim was added next day at a final volume of 10%. After 6 days, the resultant effector cells were assayed in a 4-hour 51Cr-release assay at various E:T ratios with EL4 cells (a mouse lymphoma cell line) as targets. EL4 cells (5×10⁵) were pulsed for 2 hours in 200 μl CTM, labeled with 100 μCi 51 Cr with and without 1 μM indicated peptide. Target cells were washed three times and co-incubated with effector cells at a final volume of 200 μl for 4 hours. Supernatants were harvested and counted using a gamma counter. The percentage of specific 51 Cr-release was calculated as follows: 100 X (experimental release−spontaneous release)/(maximum release−spontaneous release). Spontaneous release was determined from target cells incubated without effector cells; maximum release was determined from target cells incubated in the presence of 1% Triton X-100.

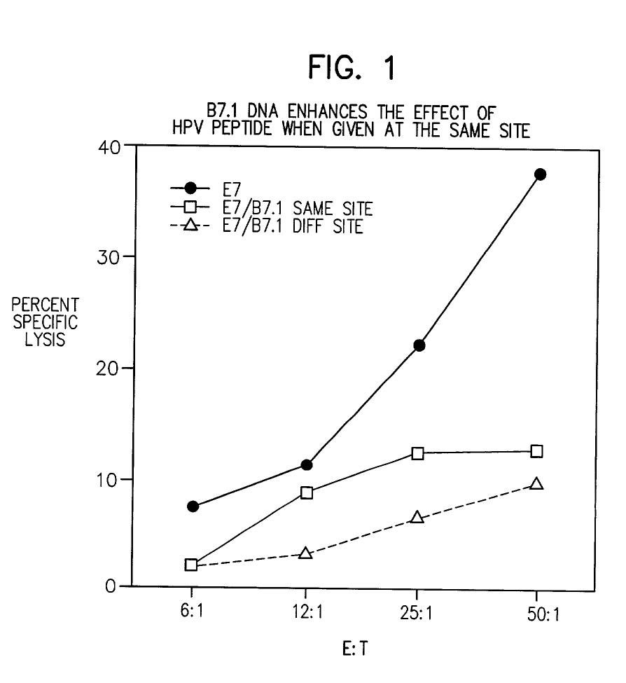

The results of the foregoing study are presented in FIG. 1. The HPV16 E7 peptide(49-57), which is an H2Db binding peptide, is immunogenic in C57BL/6 (B6) (H-2b) mice. To enhance the immunogenicity of antigen-peptide vaccines, the antigen-peptide (HPV16 E7 peptide (49-57) was coordinately administered with a DNA plasmid encoding a co-stimulatory molecule (B7-1). The plasmid was constructed to contain the full length B7-1 open reading frame driven by an exemplary, CMV promoter. When the E7 peptide was coordinately administered with the B7-1 co-stimulatory molecule DNA vector, the immunogenic effect of the peptide was increased 3-4 times in comparison to the level of CTL stimulation observed when the peptide emulsion was given without the B7-1-encoding vector. These results demonstrate that coordinate administration of a co-stimulatory molecule as DNA along with a peptide antigen yields an unexpectedly enhanced immunostimulatory effect. These effects are also expected, and will likely have a wider and improved application by utilizing multideterminant peptide or protein vaccine agents with a vector encoding a costimulatory molecule. Likewise, the methods disclosed herein may be further improved by coordinately administering a peptide or protein antigen comprising one or more antigenic determinants (i.e., T helper, CTL or B cell epitopes) with one or more vectors expressing a combination of multiple co-stimulatory molecules as disclosed herein above.

As will be understood by those persons practicing with ordinary skill in the art, the various aspects and embodiments of the invention described above can be further optimized and refined through routine evaluations and practical manipulations. In this context, while the conception and practice of the invention is clearly evinced and detailed by the above disclosure, further exemplification of the methods and compositions provided within the invention will follow a routine course of inquiry—involving the application of known methods in accordance with the teachings of this specification. Among these methods, further refinements will be achieved by testing various vaccine formulations, dosages and protocols, including a range of candidate vaccines incorporating different protein or peptide antigens and vectors encoding co-stimulatory molecules, as contemplated above, within accepted assays and animal model systems. Among these assays and model systems, a variety of assays will be employed that measure immune responses in vitro and in vivo, including CTL activation (e.g., as detected by CTL proliferation, cytokine induction and/or cytolytic assays) using various vaccine formulations and methods of the invention. Likewise, various in vivo models to determine anti-viral and anti-tumor effects elicited in live subjects by various methods and compositions of the invention will be employed to clarify equivalents and obvious modifications of the invention. For ease and timeliness of description, execution of these various additional assays, models and refinements is reserved, as the implementation of these well known and widely accepted procedures is amply disclosed elsewherein the art. A review of the following references (Khleif et al., J. Immunother. 22:155-165, 1999; Ressing et al., J. Immunol. 154:5934-5943,1995; Feltkamp et al., Eur. J. Immunol. 23:2242-2249, 1993; Zhu et al., Scand. J. Immunol. 42:557-563, 1995; Mayordomo et al., Nat. Med. 1: 1297-1302, 1995; Van Driel et al., Eur. J. Cancer 35:946-952, 1999; Ji et al., Int. J. Cancer 78:41-45, 1998; Feltkamp et al., Eur. J. Immunol. 25:2638-2642, 1995; and Kast et al., J. Imniunol. 152:3904-3912, 1994, Ressing et al., Immunotechnology 2:241-251, 1996; Melief et al., Curr. Opin. Immunol. 8:651-657, 1996; Vierboom et al., J. Immunother. 21:399-408, 1998, each incorporated herein by reference), as well as the additional references disclosed in the above description, readily conveys these models and procedures without the need for repetition herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for eliciting an immune response in a subject comprising administering an immunogenically effective amount of a peptide or protein antigen comprising one or more T cell epitope(s) coordinately with a non-viral vector comprising a polynucleotide encoding a T cell co-stimulatory molecule.

2. The method of claim 1, wherein the peptide or protein antigen comprises a T cell epitope of a tumor antigen or viral antigen.

3. The method of claim 2, wherein the tumor antigen is selected from p53, ras, rb, mcc, apc, dcc; nfl; VHL; MEN1, MEN2, MLM, Her-2neu, CEA, PSA; Muc1, Gp100, tyrosinase, or MART1.

4. The method of claim 3, wherein the tumor antigen is selected from a mutant or normal p53 or ras protein.

5. The method of claim 4, wherein the peptide antigen comprises a sequence of at least nine amino acids spanning a mutation in p53 or ras.

6. A method for eliciting an immune response in a subject comprising administering an immunogenically effective amount of a protein antigen comprising at least one T cell epitope coordinately with a non-viral vector comprising a polynucleotide encoding a T cell co-stimulatory molecule.

7. The method of claim 2, wherein the viral antigen is selected from a human immunodeficiency virus (HIV), hepatitis B virus (HBV), hepatitis C virus (HCV), herpes simplex virus (HSV) or human papilloma virus (HPV) antigen.

8. The method of claim 7, wherein the peptide antigen comprises at least nine contiguous amino acids of a HPV antigenic protein.

9. The method of claim 7, wherein the peptide antigen comprises at least nine contiguous amino acids of a HIV antigenic protein.

10. The method of claim 7, wherein the peptide antigen comprises at least nine contiguous amino acids of a HBV or HCV antigenic protein.

11. The method of claim 1, wherein the co-stimulatory molecule is selected from B7-1, B7-2, B7-3, B7-H, ICAM1, ICAM2, ICAM3, LFA1, LFA2 or LFA3.

12. The method of claim 11, wherein the co-stimulatory molecule is B7-1.

13. The method of claim 1, wherein the peptide antigen and non-viral vector encoding one or more T cell co-stimulatory molecules are administered to the subject simultaneously as a mixture in a pharmaceutically acceptable carrier or diluent.

14. The method of claim 1, wherein the peptide antigen and non-viral vector encoding the T cell co-stimulatory molecule are administered separately to the subject in a sequential vaccination protocol.

15. The method of claim 1, wherein the peptide antigen and non-viral vector encoding the T cell co-stimulatory molecule are administered to proximal target sites selected from the same, or closely-adjacent, intradermal, subcutaneous, mucosal or intratumoral sites.

16. The method of claim 1, wherein the non-viral vector is selected from a RNA or DNA vector.

17. The method of claim 1, wherein the non-viral vector comprises a naked DNA vector having the polynucleotide encoding the co-stimulatory molecule operably linked to regulatory elements necessary for expression of the co-stimulatory molecule in eukaryotic cells.

18. An immunogenic composition comprising an immunogenically effective amount of a peptide or protein antigen comprising a T cell epitope, and a non-viral vector comprising a polynucleotide that encodes a T cell co-stimulatory molecule operably linked to regulatory elements necessary for expression of the co-stimulatory molecule in eukaryotic cells, formulated in a pharmaceutically acceptable carrier or diluent.

19. The immunogenic composition of claim 18, wherein the peptide antigen comprises a T cell epitope of a tumor antigen or viral antigen.

20. The immunogenic composition of claim 19, wherein the tumor antigen is selected from p53, ras, rb, mcc, apc, dcc; nfl; VHL; MEN1, MEN2, MLM, Her-2neu, CEA, PSA; Muc1, Gp100, tyrosinase, or MART1.

21. The immunogenic composition of claim 20, wherein the peptide antigen comprises a sequence of at least nine amino acids spanning a mutation in p53 or ras.

22. The immunogenic composition of claim 18, wherein a protein antigen is administered as a purified protein or a tumor lysate component of a vaccine formulation.

23. The immunogenic composition of claim 19, wherein the viral antigen is selected from an antigenic protein of human immunodeficiency virus (HIV), hepatitis B virus (HBV), hepatitis C virus (HCV); herpes simplex virus (HSV), or human papilloma virus (HPV) antigen.

24. The immunogenic composition of claim 23, wherein the peptide antigen comprises at least nine contiguous amino acids of a HPV E6 or E7 protein.

25. The immunogenic composition of claim 23, wherein the peptide antigen comprises at least nine contiguous amino acids of a HIV antigenic protein.

26. The immunogenic composition of claim 23, wherein the peptide antigen comprises at least nine contiguous amino acids of a HBV antigenic protein.

27. The immunogenic composition of claim 18, wherein the co-stimulatory molecule is selected from B7-1, B7-2, B7-3, B7-H, ICAM 1, ICAM2, ICAM3, LFA1, LFA2 or LFA3.

28. The immunogenic composition of claim 27, wherein the co-stimulatory molecule is B7-1.

29. The immunogenic composition of claim 18, wherein the non-viral vector is selected from a RNA or DNA vector.

30. The immunogenic composition of claim 29, wherein the non-viral vector comprises a naked DNA vector having the polynucleotide encoding the co-stimulatory molecule operably linked to regulatory elements necessary for expression of the co-stimulatory molecule in eukaryotic cells.

31. The immunogenic composition of claim 18, wherein the peptide antigen comprises a cytotoxic T cell (CTL) epitope.


SEQUENCE LISTING (61 sequences)

SEQ ID NO: 1 - protein, 35 aa, Artificial Sequence
  Description of Artificial Sequence HIV PEPTIDE ANTIGEN
EQMHEDIISLWDQSLKPCVKRIQRGPGRAFVTIGK

SEQ ID NO: 2 - protein, 39 aa, Artificial Sequence
  Description of Artificial Sequence HIV PEPTIDE ANTIGEN
KQIINMWQEVGKAMYAPPISGQIRRIQRGPGRAFVTIGK

SEQ ID NO: 3 - protein, 39 aa, Artificial Sequence
  Description of Artificial Sequence HIV PEPTIDE ANTIGEN
RDNWRSELYKYKVVKIEPLGVAPTRIQRGPGRAFVTIGK

SEQ ID NO: 4 - protein, 48 aa, Artificial Sequence
  Description of Artificial Sequence HIV PEPTIDE ANTIGEN
AVAEGTDRVIEVVQGAYRAIRHIPRRIRQGLERRIQRGPGRAFVTIGK

SEQ ID NO: 5 - protein, 42 aa, Artificial Sequence
  Description of Artificial Sequence HIV PEPTIDE ANTIGEN
DRVIEVVQGAYRAIRHIPRRIRQGLERRIQRGPGRAFVTIGK

SEQ ID NO: 6 - protein, 30 aa, Artificial Sequence
  Description of Artificial Sequence HIV PEPTIDE ANTIGEN
DRVIEVVQGAYRAIRRIQRGPGRAFVTIGK

SEQ ID NO: 7 - protein, 31 aa, Artificial Sequence
  Description of Artificial Sequence HIV PEPTIDE ANTIGEN
AQGAYRAIRHIPRRIRRIQRGPGRAFVTIGK

SEQ ID NO: 8 - protein, 35 aa, Artificial Sequence
  Description of Artificial Sequence HIV PEPTIDE ANTIGEN
EQMHEDIISLWDQSLKPCVKRIHIGPGRAFYTTKN

SEQ ID NO: 9 - protein, 39 aa, Artificial Sequence
  Description of Artificial Sequence HIV PEPTIDE ANTIGEN
KQIINMWQEVGKAMYAPPISGQIRRIHIGPGRAFYTTKN

SEQ ID NO: 10 - protein, 39 aa, Artificial Sequence
  Description of Artificial Sequence HIV PEPTIDE ANTIGEN
RDNWRSELYKYKVVKIEPLGVAPTRIHIGPGRAFYTTKN

SEQ ID NO: 11 - protein, 48 aa, Artificial Sequence
  Description of Artificial Sequence HIV PEPTIDE ANTIGEN
AVAEGTDRVIEVVQGAYRAIRHIPRRIRQGLERRIHIGPGRAFYTTKN

SEQ ID NO: 12 - protein, 43 aa, Artificial Sequence
  Description of Artificial Sequence HIV PEPTIDE ANTIGEN
DRVIEVVQGAYRAIRHIPRRIRQGLERRIHIGPGRAFYTTKIN

SEQ ID NO: 13 - protein, 30 aa, Artificial Sequence
  Description of Artificial Sequence HIV PEPTIDE ANTIGEN
DRVIEVVQGAYRAIRRIHIGPGRAFYTTKN

SEQ ID NO: 14 - protein, 31 aa, Artificial Sequence
  Description of Artificial Sequence HIV PEPTIDE ANTIGEN
AQGAYRAIRHIPRRIRRIHIGPGRAFYTTKN

SEQ ID NO: 15 - protein, 15 aa, Artificial Sequence
  Description of Artificial Sequence HIV PEPTIDE ANTIGEN
RIQRGPGRAFVTIGK

SEQ ID NO: 16 - protein, 10 aa, Artificial Sequence
  Description of Artificial Sequence HIV PEPTIDE ANTIGEN
RGPGRAFVTI

SEQ ID NO: 17 - protein, 10 aa, Artificial Sequence
  Description of Artificial Sequence HIV PEPTIDE ANTIGEN
IGPGRAFYTT

SEQ ID NO: 18 - protein, 10 aa, Artificial Sequence
  Description of Artificial Sequence HIV PEPTIDE ANTIGEN
IGPGRAFYAT

SEQ ID NO: 19 - protein, 10 aa, Artificial Sequence
  Description of Artificial Sequence HIV PEPTIDE ANTIGEN
KGPGRVIYAT

SEQ ID NO: 20 - protein, 10 aa, Artificial Sequence
  Description of Artificial Sequence HIV PEPTIDE ANTIGEN
IGPGRAFHTT

SEQ ID NO: 21 - protein, 10 aa, Artificial Sequence
  Description of Artificial Sequence HIV PEPTIDE ANTIGEN
IGPGRTLYAR

SEQ ID NO: 22 - protein, 10 aa, Artificial Sequence
  Description of Artificial Sequence HIV PEPTIDE ANTIGEN
LGPGRVWYTT

SEQ ID NO: 23 - protein, 10 aa, Artificial Sequence
  Description of Artificial Sequence HIV PEPTIDE ANTIGEN
IGPGRAFRTR

SEQ ID NO: 24 - protein, 15 aa, Artificial Sequence
  Description of Artificial Sequence HIV PEPTIDE ANTIGEN
RIQRGPGRAFVTIGK

SEQ ID NO: 25 - protein, 15 aa, Artificial Sequence
  Description of Artificial Sequence HIV PEPTIDE ANTIGEN
RIHIGPGRAFYTTKN

SEQ ID NO: 26 - protein, 15 aa, Artificial Sequence
  Description of Artificial Sequence HIV PEPTIDE ANTIGEN
SITKGPGRVIYATGQ

SEQ ID NO: 27 - protein, 15 aa, Artificial Sequence
  Description of Artificial Sequence HIV PEPTIDE ANTIGEN
SIHIGPGPAFYATGD

SEQ ID NO: 28 - protein, 15 aa, Artificial Sequence
  Description of Artificial Sequence HIV PEPTIDE ANTIGEN
SLSIGPGRAFRTREI

SEQ ID NO: 29 - protein, 15 aa, Artificial Sequence
  Description of Artificial Sequence HIV PEPTIDE ANTIGEN
SISIGPGRAFFATTD

SEQ ID NO: 30 - protein, 15 aa, Artificial Sequence
  Description of Artificial Sequence HIV PEPTIDE ANTIGEN
SIYIGPGRAFHTTGR

SEQ ID NO: 31 - protein, 15 aa, Artificial Sequence
  Description of Artificial Sequence HIV PEPTIDE ANTIGEN
GIAIGPGRTLYAREK

SEQ ID NO: 32 - protein, 15 aa, Artificial Sequence
  Description of Artificial Sequence HIV PEPTIDE ANTIGEN
RVTIGPGRVWYTTGE

SEQ ID NO: 33 - protein, 15 aa, Artificial Sequence
  Description of Artificial Sequence HIV PEPTIDE ANTIGEN
SIRIGPGKVFTAKGG

SEQ ID NO: 34 - protein, 15 aa, Artificial Sequence
  Description of Artificial Sequence HIV PEPTIDE ANTIGEN
GIHFGPGQALYTTGI

SEQ ID NO: 35 - protein, 14 aa, Artificial Sequence
  Description of Artificial Sequence HIV PEPTIDE ANTIGEN
STPIGLGQMYTTRG

SEQ ID NO: 36 - protein, 15 aa, Artificial Sequence
  Description of Artificial Sequence HIV PEPTIDE ANTIGEN
STPIGLGQALYTTRI

SEQ ID NO: 37 - protein, 15 aa, Artificial Sequence
  Description of Artificial Sequence HIV PEPTIDE ANTIGEN
RTPTGLGQSLYTTRS

SEQ ID NO: 38 - protein, 14 aa, Artificial Sequence
  Description of Artificial Sequence HIV PEPTIDE ANTIGEN
QIDSKLREQFGNNK

SEQ ID NO: 39 - protein, 20 aa, Artificial Sequence
  Description of Artificial Sequence HIV PEPTIDE ANTIGEN
GSDTITLPCRIKQFINMWQE

SEQ ID NO: 40 - protein, 15 aa, Artificial Sequence
  Description of Artificial Sequence HIV PEPTIDE ANTIGEN
NYTSLIHSLIEESQN

SEQ ID NO: 41 - protein, 15 aa, Artificial Sequence
  Description of Artificial Sequence HIV PEPTIDE ANTIGEN
EQELLELDKWASLWN

SEQ ID NO: 42 - protein, 15 aa, Artificial Sequence
  Description of Artificial Sequence HIV PEPTIDE ANTIGEN
RIVELLGRRGWEALK

SEQ ID NO: 43 - protein, 25 aa, Artificial Sequence
  Description of Artificial Sequence HIV PEPTIDE ANTIGEN
IETVPVKLKPGMDGPKVKQWPLTEE

SEQ ID NO: 44 - protein, 25 aa, Artificial Sequence
  Description of Artificial Sequence HIV PEPTIDE ANTIGEN
AIFQSSMTKILEPFRKQNPDIVIYQ

SEQ ID NO: 45 - protein, 25 aa, Artificial Sequence
  Description of Artificial Sequence HIV PEPTIDE ANTIGEN
NPDIVIYQYMDDLYVGSDLEIGQHR

SEQ ID NO: 46 - protein, 25 aa, Artificial Sequence
  Description of Artificial Sequence HIV PEPTIDE ANTIGEN
DLEIGQHRTKIEELRQHLLRWGLTT

SEQ ID NO: 47 - protein, 25 aa, Artificial Sequence
  Description of Artificial Sequence HIV PEPTIDE ANTIGEN
PLTEEAELELAENREILKEPVHGVY

SEQ ID NO: 48 - protein, 25 aa, Artificial Sequence
  Description of Artificial Sequence HIV PEPTIDE ANTIGEN
EIQKQGQGQWTYQIYQEPFKNLKTG

SEQ ID NO: 49 - protein, 9 aa, Artificial Sequence
  Description of Artificial Sequence HIV PEPTIDE ANTIGEN
NPDIVIYQY

SEQ ID NO: 50 - protein, 9 aa, Artificial Sequence
  Description of Artificial Sequence HIV PEPTIDE ANTIGEN
FPVRPQVPL

SEQ ID NO: 51 - protein, 9 aa, Artificial Sequence
  Description of Artificial Sequence HIV PEPTIDE ANTIGEN
TPPLVKLWY

SEQ ID NO: 52 - protein, 10 aa, Artificial Sequence
  Description of Artificial Sequence HIV PEPTIDE ANTIGEN
EPIVGAETFY

SEQ ID NO: 53 - protein, 9 aa, Artificial Sequence
  Description of Artificial Sequence HIV PEPTIDE ANTIGEN
SPAIFQSSM

SEQ ID NO: 54 - protein, 9 aa, Artificial Sequence
  Description of Artificial Sequence HIV PEPTIDE ANTIGEN
YPLTFGWCF

SEQ ID NO: 55 - protein, 9 aa, Artificial Sequence
  Description of Artificial Sequence HIV PEPTIDE ANTIGEN
EPIVGAETF

SEQ ID NO: 56 - protein, 10 aa, Artificial Sequence
  Description of Artificial Sequence HIV PEPTIDE ANTIGEN
VPLDKDFRKY

SEQ ID NO: 57 - protein, 11 aa, Artificial Sequence
  Description of Artificial Sequence HIV PEPTIDE ANTIGEN
RPQVPLRPMTY

SEQ ID NO: 58 - protein, 9 aa, Artificial Sequence
  Description of Artificial Sequence HIV PEPTIDE ANTIGEN
IPLTEEAEL

SEQ ID NO: 59 - protein, 9 aa, Artificial Sequence
  Description of Artificial Sequence HIV PEPTIDE ANTIGEN
DPNPQEVVL

SEQ ID NO: 60 - protein, 9 aa, Artificial Sequence
  Description of Artificial Sequence HIV PEPTIDE ANTIGEN
RPIVSTQLL

SEQ ID NO: 61 - protein, 9 aa, Artificial Sequence
  Description of Artificial Sequence HIV PEPTIDE ANTIGEN
LPCRIKQII